(19) United States
(12) Patent Application Publication
(10) Pub. No.: US 2016/0243218 A1
(43) Pub. Date: Aug. 25, 2016

(54) INHIBITION OF NONSENSE MEDIATED DECAY PATHWAYS

(71) Applicant: UNIVERSITY OF MIAMI, Miami, FL (US)

(72) Inventor: Eli Gilboa, Coral Gables, FL (US)

(21) Appl. No.: 14/982,734

(22) Filed: Dec. 29, 2015

Related U.S. Application Data
(63) Continuation of application No. 13/810,808, filed on May 3, 2013, now abandoned.
(60) Provisional application No. 61/365,812, filed on Jul. 20, 2010.

(51) Int. Cl. A61K 39/385; A61K 47/48

(52) U.S. Cl. CPC A61K 39/385; A61K 47/48246

(57) ABSTRACT

Compositions for inducing or enhancing antigenicity of a target cell by modulating the nonsense mediated decay pathway in the target cell. The compositions comprise one or more cell binding ligands providing specificity and delivery of an oligonucleotide or other molecule to the target. These compositions have broad applicability in the treatment of many diseases.

### CROSS-REFERENCE TO RELATED APPLICATIONS

This application claims priority to U.S. Provisional Application No. 61/365,812, filed Jul. 20, 2010, the entire contents of which are incorporated herein by reference.

### FIELD OF THE INVENTION

Embodiments of the invention provide compositions and methods for highly selective targeting of heterologous nucleic acid sequences or other molecules which modulate the expression or function of nonsense mediated decay pathways. The heterologous nucleic acid sequences comprise oligonucleotides, for example, short interfering RNA's (siRNA's) which are targeted to desired cells in vivo, such as cells of the tumor stroma, and which bind in a sequence dependent manner to their targets.

### BACKGROUND

Many therapeutic agents have been made available in the treatment of diseases such as cancer. There have been many challenges, however, in obtaining therapies which are effective. The important challenges facing many therapeutics in treatment of cancer include: (i) Metastatic disease, which is often undetectable and/or inaccessible, not the primary lesion, is the primary cause of death among cancer patients. The treatment, therefore, has to access disseminated disease. (ii) The drug has to be targeted to the tumor lesions in the cancer patient. Expression of new products in nontransformed cells will expose normal tissue to the effects of the drug which can be toxic to the cells and the patient. (iii) The therapy should be clinically feasible, from the standpoint of cost, regulatory approval process, and complexity of treatment. For example, delivery and expression of “foreign” genes in tumor cells can be achieved using Adeno- or poxvirus-based vectors. However, poor tumor penetrance, their potential immunogenicity, and the challenges associated with using viral vectors in clinical settings, has precluded their use for this purpose. (iv) The therapy should be broadly useful for all types of cancer and cancer patients.

### SUMMARY

Embodiments of the invention comprise the generation of multi-domain molecules comprising a target specific domain and at least one domain, which modulates expression and function of molecules associated with nonsense mediated decay pathways.

Methods of treating a patient comprise administration of a therapeutically effective amount of multi-domain biologically active molecules. In a preferred embodiment, the multi-domain molecules comprise a cell binding ligand which binds to cells in the tumor stroma (such as, for example, endothelial cells, fibroblasts or immune cells) for specifically targeting an oligonucleotide, e.g. interference RNA (RNAi) to a desired cell in vivo. The cell binding ligands are generated against specific products expressed by a target cell, for example, integrins, glucose-regulated protein 78, neuropilin, growth factor receptors, e.g. VEGF receptors, and the oligonucleotides are specific for inhibiting the nonsense mediated decay pathway and associated molecules. Inhibition of the nonsense mediated decay pathway allows for the up-regulation of existing antigens and/or the induction of new antigens not previously expressed on the target cells and/or novel antigens which results in the induction or enhancement of antigen city of a the target cell ultimately leading to its destruction by the immune system.

In preferred embodiments, a composition for inhibiting nonsense mediated decay (NMD) pathways in patients in vivo comprises at least one first domain which specifically binds to at least one tumor cell target or normal cell target in a tumor stroma and at least one second domain specific for a molecular component of nonsense mediated decay pathways, wherein the second domain comprises an antisense oligonucleotide molecule, peptides, proteins, nucleic acids, organic or inorganic molecules, which inhibit the nonsense mediated decay pathway.

In some embodiments, the oligonucleotide molecule of the second domain, comprises at least one of a short interfering RNA (siRNA); a micro-interfering RNA (miRNA); antisense oligonucleotides; a small, temporal RNA (stRNA); a short, hairpin RNA (shRNA), or combinations thereof.

In a preferred embodiment, the oligonucleotide molecule of the second domain, inhibits function and/or expression of at least one factor associated with the NMD pathway comprising at least one of: RENT1, RENT2, eIF4A, UPF1, UPF2, UPF3B, RNPS1, Y14, MAGOH, NMD1, SMG, or combinations thereof.

In a preferred embodiment, the first domain specifically or selectively binds to any target desired. Preferably, the target is a tumor cell target, a normal cell target, cells in tumor stroma or combinations thereof. Preferably, the first domain specifically binds to tumor or normal cell targets comprising: vascular endothelial growth factor (VEGF), vascular endothelial growth factor receptor (VEGFR-2), Tie2; fibronectin, vitronectin, collagen, laminin, fibroblast antigens, fibroblast activation protein (FAP), glucose-regulated protein 78 (GRP78), stromal derived factor 1 (SDF-1), MCP-1, MIP-1α, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, neurophilin, MAdCAM-1, neuropilin-1, α4 intcgrins, α5 intcgrins, or beta defensins 3 and 4.

The molecules which specifically bind to the targets can be any type of molecule that will endow the composition with specificity. Thus, in preferred embodiments, the molecules comprising the first domain comprise at least one of: peptides having a C-terminal arginine (CendR) motif or C-terminal lysine motif, glucose-regulated protein 78 (GRP78) binding peptides, integrin binding peptides, Pie42 peptides, aptamers, antibodies, antibody fragments or combinations thereof.

In some embodiments, the molecules of the first domain can be peptides and these peptides of first domain comprise at least one peptide set forth as SEQ ID NOS: 1, 2, 3, 4, 5 or combinations thereof.

In other embodiments, the first and second domains further comprise one or more domains to promote intracellular delivery, cytoplasmic delivery, bioavailability, or combinations thereof. Examples include at least one of: polylysine, polyarginine, Antennapedia-derived peptides, HIV derived tat peptide, a fusogenic peptide from influenza hemagglutinin protein, a 9mer Arg oligopeptide, peptide transporters, intracellular localization domain sequences, or combinations thereof.

In one embodiment, the one or more domains promoting bioavailability comprise at least one of: cholesterol, polyethylene glycol, or combinations thereof.

In another preferred embodiment, the first domain is an aptamer specific for the target molecules and the second domain is an siRNA specific for an NMD target.

In one embodiment, the at least one first domain is fused or linked to each other and/or with the second domain by at least one linker molecule. The linker molecule preferably comprises nucleotide, non-nucleotide, or mixed nucleotide/non-nucleotide molecules. These can be about 2 nucleotides length up to about 50 nucleotides in length.

In one embodiment, the non-nucleotide linker comprises abasic nucleotide, polyether, polyamine, polyamide, peptide, carbohydrate, lipid, polyhydrocarbon, polymeric compounds or combinations thereof, having one or more monomeric units.

In other preferred embodiment, a composition for inducing novel antigens in abnormal cells, comprising: a multi-domain molecule having at least one target specific domain and at least one domain, which modulates expression and function of molecules associated with nonsense mediated decay pathways.

In another preferred embodiment, a method of inducing or enhancing immunogenicity of a target cell in vitro or in vivo and modulating an antigen specific immune response in patient comprising: obtaining a composition comprising at least target binding molecule conjugated to at least one oligonucleotide molecule wherein the target binding ligand selectively binds to tumor cell or normal cell target ligands of tumor stroma and the oligonucleotide is specific for a molecule associated with at least one factor associated with a nonsense mediated decay pathway (NMD); administering the composition in a therapeutically effective amount to the patient.

The oligonucleotide molecule of the second domain inhibits at least one factor associated with the NMD pathway comprising at least one of: RENT1, RENT2, UPF1, UPF2, UPF3B, RNPS1, Y14, MAGOH, NMD1 or SMG. The target binding ligand selectively binds at least one of: vascular endothelial growth factor (VEGF), vascular endothelial growth factor receptor (VEGFR-2), Tie2; fibronectin, vitronectin, collagen, laminin, fibroblast antigens, fibroblast activation protein (FAP), glucose-regulated protein 78 (GRP78), stromal derived factor 1 (SDF-1), MCP-1, MIP-1α, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, neuropilinMAdCAM-1, neuropilin-1, α4 integrins, α5 integrins, or beta defensins 3 and 4.

In some embodiments, the target binding ligand comprises at least one peptide having a C-terminal arginine (CendR) motif or C-terminal lysine motif, glucose-regulated protein 78 (GRP78) binding peptides, integrin binding peptides, or Pie42 peptides. In some aspects, the target binding ligands comprising the first domain comprise at least one peptide set forth as SEQ ID NOS: 1, 2, 3, 4, 5, 6, 7, 8 or combinations thereof.

In some embodiments, the target binding ligand comprises at least one motif: RGD, R/KXXR/K, CXN+1CX N+1CXN+1C motif, wherein X is a variable amino acid and n=0 to 20, variants, mutants or analogs thereof.

In some embodiments, novel target binding ligands comprise peptides which bind to tumor vasculature or tumor stroma are identified by phage display libraries or high-throughput screens.

In a preferred embodiment, the target binding ligand is an aptamer.

In one embodiment, an immune co-stimulatory agent is co-administered with the NMD inhibitory agent. An example of an immune co-stimulatory agent is 4-1BB.

In one embodiment, a method of up-regulating existing and/or inducing new or novel antigens on a cell's surface comprise contacting the cell with the composition comprising at least one first domain which binds to at least one tumor or normal cell target ligand in tumor stroma and at least one second domain specific for a molecular component of nonsense mediated decay pathways, wherein the NMD domain comprises an antisense oligonucleotide molecule, peptides, proteins, nucleic acids, organic or inorganic molecules which inhibit the nonsense mediated decay pathway.

In another preferred embodiment, a method of preventing or treating cancer in vivo, comprising administering to a patient a therapeutically effective amount of a composition comprising at least one first domain which binds to at least one tumor cell or normal cell target ligand in tumor stroma and at least one second domain specific for a molecular component of nonsense mediated decay pathways, wherein the NMD domain comprises an antisense oligonucleotide molecule, peptides, proteins, nucleic acids, organic or inorganic molecules which inhibit the nonsense mediated decay pathway.

In all embodiments, an immune co-stimulatory agent comprising an aptamer and immune cell stimulatory agent may also be included as part of the therapeutic regimen. Examples of immune cell stimulatory molecules include without limitation: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

Other aspects are described infra.

### BRIEF DESCRIPTION OF THE DRAWINGS

FIG. 1 is a schematic representation showing the mechanism by which the NMD process prevents the accumulation of premature termination codon (PTC) containing mRNAs in eukaryotic cells. Removal of introns from the pre-mRNA leaves behind an exon junction complex (EJC) demarcating the splice junctions (Panel A). An NMD complex consisting of several factors including Upf1, Upf2 and Up3 is then assembled on each EJC as shown in Panel B. SMG1, which phosphorylates Upf1, and Upf1 are the two key rate limiting factors in the formation of the complex. When the mRNA undergoes the first round of translation, called the “pioneer translation”, the EJC/NMD complex is removed, presumably as a result of the translational machinery moving thru the region, thereby rendering the mRNA stable and competent for additional rounds of translation. If a PTC is present in an exon (other than the last exon), for example as a result of an inframe nonsense mutation, the EJC/NMD complexes downstream to the PTC are not removed from the mRNA. The attached NMD complex then triggers the degradation of the mRNA.

FIG. 2A-2C shows the expression of Upf2 or Smg1 shRNA in CT26 tumor cells leads to immune-mediated inhibition of tumor growth. FIG. 2A: intratumoral accumulation of OVA-specific OT-I T cells in response to NMD inhibition. B16/F10 tumor cells transduced with shRNA-encoding lentiviral vectors were stably transfected with an NMD reporter plasmid containing the class I-restricted epitope of chicken ovalbumin (OVA). Mice were implanted subcutaneously with parental tumor cells (wild-type (WT) B16) or with the lentivirus-transduced tumor cells, and either received or did not receive doxycycline in their drinking water. When tumors became palpable, mice were injected with either OT-I or Pmel-1 transgenic CD8⁺ T cells (three mice per group). Six days later, tumors were excised and analyzed for OT-I and Pmel-1 T-cell content by flow cytometry. Ctrl, control. n=2. FIG. 2B: Balb/c mice were implanted subcutaneously with CT26 tumor cells stably transduced with the shRNA inducible lentiviral vector encoding Smg1, Upf2 and control shRNA (ten mice per group). Each group was divided into two subgroups receiving (filled circles) or not receiving (open circles) doxycycline in the drinking water. n=2. FIG. 2C: same as FIG. 2B except that tumor cells were injected into immune-deficient nude mice. n=1.

FIG. 3A-3C show the inhibition of tumor growth in mice treated with PSMA aptamer targeted Upf2 and Smg1 siRNAs. FIG. 3A: Balb/c mice were implanted subcutaneously with PSMA-CT26 tumor cells and 3 days later injected via the tail vein with PBS (filled circles) or with PSMA aptamer-siRNA conjugates (open circles, control siRNA; open squares, Upf2 siRNA; filled squares, Smg1 siRNA) (5 mice per group). n=2. FIG. 3B: C57BL/6 mice were implanted with PSMA-B16/F10 tumor cells by tail vein injection, and 5 days later were injected with PSMA aptamer-siRNA conjugates (ten mice per group). Metastatic load was determined by measuring lung weight at the time of euthanization. n=2. FIG. 3C: combination immunotherapy using NMD inhibition and 4-1BB co-stimulation. PSMA-CT26 tumor-bearing mice (five mice per group) were treated with various combinations of PSMA aptamer conjugated to Smg1 or control siRNA and an agonistic or co-stimulation-deficient 4-1BB aptamer dimer (mut4-1BB) and monitored for tumor growth. n=1.

FIGS. 4A-4C show the PSMA aptamer-Smg1 siRNA rejection of PSMA-expressing, but not parental, CT26 tumor cells. FIG. 4A: mice were co-implanted subcutaneously with PSMA-expressing (left flank) and parental (right flank) CT26 tumor cells and injected with PSMA aptamer-Smg1 siRNA via the tail vein. FIG. 4B: fifteen days after tumor inoculation, ³²P-labelled aptamer-siRNA was injected, and 3 or 24 h later tumors were excised and the ³²P content determined. n=3. FIG. 4C: three days after tumor inoculation, mice were injected with aptamer-siRNA conjugate (eight mice per group) as described in FIG. 3A and tumor growth was monitored. Open circles, parental CT26; filled circles, PSMA-CT26. n=2.

FIG. 5 shows the comparison of PSMA aptamer-Smg1 siRNA treatment to vaccination with GM-CSF expressing irradiated tumor cells. C57BL/6 mice were injected intravenously with B16/F10 tumor cells and treated with PSMA aptamer-siRNA conjugates starting at day 5 as described in FIG. 3B, or vaccinated with GM-C SF-expressing irradiated B16/F10 tumor cells (GVAX) starting at days (D) 1 or 5. n=1.

FIGS. 6A, 6B show RNA downregulation and NMD inhibition in CT26 tumor cell stably expressing Upf-2 or SMG-1 shRNA. Colon carcinoma-derived CT26 tumor cells were stably transduced with the PTIG-U6tetO lentiviral vector encoding Upf-2 and SMG-1 shRNA. PTIG-U6tetOshRNA contains a U6-promoter driven shRNA cassette which is under tet regulation (FIG. 6A), as well as a bicistronic CMV promoter driven cassette encoding the tet repressor and EGFP. Stably transduced cells were isolated by sorting for EGFP cells and grown presence or absence of doxycyclinc. FIG. 6A: expression of Upf-2 or SMG-1 RNA is reduced in CT26 tumor cells expressing the corresponding shRNAs (culturing tumor cells in the presence of doxycycline), but not parental CT26 tumor cells. The relative amounts of actin, Upf2 and SMG-1 RNA were determined by semiquantitative RT-PCR using limited cycles of amplification. FIG. 6B: shRNA-mediated Upf-2 or SMG-1 downregulation leads to NMD inhibition. NMD activity in the shRNA-expressing CT26 cells was determined by transiently transfecting the Upf-2 and SMG-1 shRNA-expressing CT26 cells with an NMD reporter plasmid encoding a β-globin gene which contains a premature termination codon (PTC) in its second exon (see diagram) or with a similar plasmid encoding the wild type (wt) β-globin gene. Cells were grown in the presence or absence of doxycycline and the relative amounts of β-globin transcripts were determined by semiquantitative RT-PCR. Cells transfected with the PTC-containing, but not wild type, β-globin gene accumulated reduced levels of globin transcripts due to NMD. However, when Upf-2 or SMG-1 shRNA expression was induced by growing cells in the presence of doxycycline leading to downregulation of its RNA as shown in FIG. 6A, expression of the PTC-containing globin transcripts is upregulated, consistent with inhibition of NMD.

FIG. 7 shows that delaying SMG-1 shRNA expression in tumor bearing mice diminishes tumor inhibition. Balb/c mice were implanted subcutaneously with the shRNA inducible lentiviral vector encoding SMG-1 shRNA as described in FIG. 2B. As indicated, doxycycline was provided in the drinking water at the day of tumor implantation (day 0) or delayed for 2, 4 or 6 days (5 mice per group).

FIGS. 8A-8C show the induction of T cell responses against NMD-controlled products. Mice were immunized with CT26 tumor cells transduced with the doxycycline-inducible SMG-1 (SMG-1) or control (Control) shRNA (FIG. 6A) in the presence doxycycline in the drinking water, and 5 or 30 days later splenocytes were isolated and enriched for either total CD3⁺ T cells, CD8⁺ T cells, or CD4⁺ T cells. FIG. 8A: induction of immune responses against tumor cells in which NMD was inhibited. Tumor antigens in the form of mRNA were isolated from SMG-1 (SMG-1) or control (Control) shRNA transduced CT26 tumor cells cultured in the presence of doxycycline, and transfected into syngeneic dendritic cells (DC). T cells isolated 5 days after tumor implantation (responders) were incubated with the mRNA transfected DC (stimulators), and proliferation was determined 4 days later by measuring ³H-thymidine incorporation. Only T cells from mice immunized with NMD-inhibited tumor cells (doxycycline in the drinking water) stimulated with DC transfected with mRNA derived from tumor cells in which NMD was inhibited (cultured in the presence of doxycycline) exhibited statistically significant enhanced proliferation. (Three experiments). FIG. 8B: no induction of immune responses against normal tissues. Total CD3⁺ T cells from mice immunized with CT26 tumor cells stably expressing SMG-1 shRNA (doxycycline in the drinking water) were incubated with DC transfected with mRNA isolated from liver, colon and prostate (Control—no mRNA) and proliferation was measured. (One experiment). (Note, the use of mRNA transfected DC as stimulators provides a useful way to compare the antigenicity of diverse cell populations and tissues that often exhibit significant variations in their background immunogenicity, e.g., stimulation of T cell proliferation measured by ³Hthymidine incorporation). FIG. 8C: epitope spread—induction of immune responses against the parental tumor. Experimental conditions as described in FIG. 8A using total CD3⁺ T cells as responders, except that T cells were isolated 5 day as well as 30 days post tumor implantation.

FIG. 9 is a schematic representation showing a primary sequence and computer generated secondary structure of PSMA aptamer-siRNA conjugates. PSMA aptamer-siRNA fusions were generated corresponding to the SWAP configuration except that two thymidine nucleotides were added to the 3′ of the passenger strand to prevent dicer binding. RNAstructre 4.1 program was used for secondary structure analysis. PSMA aptamer—black; siRNA guide strand—blue; siRNA passenger strand—red.

FIG. 10 shows the binding and uptake of PSMA aptamer-SMG-1 siRNA by PSMA-expressing CT26 tumor cells. Parental and PSMA-expressing CT26 tumor cells were incubated with anti-PSMA antibody (green) or Cy3-conjugated PSMA aptamer-SMG-1 siRNA (pink) and analyzed by confocal microscopy (60× magnification). Nuclei were stained with DAPI (blue). (Three experiments).

FIG. 11 shows the PSMA-dependent inhibition of SMG-1 or Upf-2 RNA in PSMA-CT26 tumor cells incubated with PSMA aptamer-siRNA conjugates. PSMA-CT26 or parental CT26 cells were incubated with SMG-1 siRNA, control siRNA, PSMA aptamer-SMG-1 siRNA or PSMA aptamer-control siRNA (left panel), or with PSMA aptamer-Upf-2 siRNA and PSMA-control siRNA (right panel), in the presence or absence of lipofectamine, as shown. SMG-1 and Upf-2 RNA content was determined 48 hours later using semiquantitative RT-PCR. The SMG-1 siRNA retains its function when conjugated to the PSMA aptamer since free and conjugated siRNA transfected in the presence of lipofectamine were comparably effective in downregulating SMG-1 RNA (left panel). Inhibition by the aptamer conjugated siRNAs is PSMA dependent since in the absence of transfection agent, incubation of PSMA-expressing, but not parental, CT26 tumor cells with either PSMA aptamer-SMG-1 or Upf-2 siRNA conjugates led to downregulation of its target RNA. (Two experiments).

FIGS. 12A-12B show that inhibition of tumor growth and survival of CT26 tumor bearing mice injected with PSMA-SMG-1 siRNA conjugates. Balb/c mice were implanted subcutaneously with PSMA-CT26 tumor cells and 3 days later injected via the tail vein with PBS, PSMA aptamer-control siRNA conjugate (Con), or with PSMA aptamer-SMG-1 siRNA conjugate. PSMA aptamer-SMG-1 siRNA conjugate was administered at a dose of 400 pmoles per injection on days 3, 5, 7, 9, 11, and 13, as used in the manuscript FIGS. 3A and 4C, (SMG-1 (1×)), or at a dose of 800 pmoles per injection administered at days 3, 5, 7, 9, 11, 13, 15 and 17, (SMG-1(2×). FIG. 12A: Survival:—the long term surviving mice (>40 days) had no evidence of tumor. Statistical analysis: SMG-1 (1×) versus PBS, p=0.0002; SMG-2 (2×) versus SMG-1, p=0.0327. FIG. 12B: Tumor size.

FIG. 13 shows the PSMA aptamer-SMG-1 siRNA rejection of PSMA-expressing, but not parental, CT26 tumor cells—Tumor size at day of sacrifice. Mice were sacrificed at day 19 when tumors in the PBS group reached maximum allowable size (12 mm diameter). Only the PSMA-CT26 tumors in mice treated with PSMA-SMG-1 siRNA have shown consistently diminished growth compared to contralaterally implanted parental CT26 tumors; in this experiment in 7 out of 8 mice the PSMA-CT26 tumors were either smaller than the parental CT26 tumors or completely regressed. (Two experiments).

FIG. 14 is a graph showing the inhibitory activity of peptide conjugated siRNA. Unconjugated SMG-1 siRNA (siRNA), scrambled siRNA (control), PSMA aptamer conjugated siRNA (PSMA-siRNA) and CendR or GRP78 peptide conjugated siRNA were tested for siRNA inhibition of target sequences using the dual luciferase PSICHECK® assay.

FIG. 15 is a graph showing the inhibition of metastasis in tumor bearing mice treated with CendR peptide-SMG-1 siRNA conjugate targeted to the tumor vasculature. 5 days post tumor inoculation, PSMA-expressing B16/F10 melanoma tumor bearing mice (4 mice per group) were injected i.v. 5 times at 2 day intervals with PSMA aptamer or CendR peptide conjugated to SMG-1 siRNA, (0.5 nmole and 2.5 nmole per injection, respectively). Lung metastasis was determined on day 28 by measuring lung weights. In both treatment groups, inhibition of metastasis was statistically significant despite the small number of animals per group.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or coneurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of coneurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

The term “targeting agent” refers to a molecule which specifically binds to another molecule. For example, an antibody or fragments thereof, aptamers, RGD peptides, integrins, receptors or ligands, or any other molecule that can specifically bind to a target molecule.

The term “specifically binds” to a target molecule, such as for example, an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. For example, an antibody “specifically binds” or “preferentially binds” to a target if it binds with greater affinity, avidity, more readily, and/or with greater duration than it binds to other substances. It is also understood by reading this definition that; for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide,” “siRNA,” “siRNA oligonucleotide,” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogstcen or reverse Hoogstcen types of base pairing, or the like.

The oligonucleotide may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N₆-methyladenine, 7-dcazaxanthine, 7-dcazaguanine, N₄,N₄-ethanocytosin, N₆,N₆-ethano-2,6-diaminopurine, 5-methylcytosine, 5-(C₃-C₆)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulme, J. J., Nature Biotechnology 19:17-18 (2001); Manoharan M., Biochemica et Biophysica Acta 1489:117-139 (1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000)); 2′-O, 3′-C-linked [3.2.0]bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the term “mRNA” means the presently known mRNA transcript(s) of a targeted gene, and any further transcripts which may be elucidated.

By “desired RNA” molecule is meant any foreign RNA molecule which is useful from a therapeutic, diagnostic, or other viewpoint. Such molecules include antisense RNA molecules, decoy RNA molecules, enzymatic RNA, therapeutic editing RNA and agonist and antagonist RNA.

By “antisense RNA” is meant a non-enzymatic RNA molecule that binds to another RNA (target RNA) by means of RNA-RNA interactions and alters the activity of the target RNA (Eguchi et al., 1991 Annu Rev. Biochcm. 60, 631-652).

RNA interference “RNAi” is mediated by double stranded RNA (dsRNA) molecules that have sequence-specific homology to their “target” nucleic acid sequences (Caplen, N. J., et al., Proc. Natl. Acad. Sci. USA 98:9742-9747 (2001)). In certain embodiments of the present invention, the mediators of RNA-dependent gene silencing are 21-25 nucleotide “small interfering” RNA duplexes (siRNAs). The siRNAs are derived from the processing of dsRNA by an RNase enzyme known as Dicer (Bernstein, E., et al., Nature 409:363-366 (2001)). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al., Nature 409:363-366 (2001); Boutla, A., et al., Curr. Biol. 11:1776-1780 (2001)). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

By “enzymatic RNA” is meant an RNA molecule with enzymatic activity (Cech, 1988 J. American. Med. Assoc. 260, 3030-3035). Enzymatic nucleic acids (ribozymes) act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through base-pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is meant to be a specific example. Those in the art will recognize that this is but one example, and other embodiments can be readily generated using techniques generally known in the art.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. As the target sequences are generally present in excess, at Tm, 50% of the oligonucleotides are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

The term “stringent conditions” refers to conditions under which an oligonucleotide will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. ^{3rd} ed. Cold Spring Harbor Laboratory Press: Cold Spring Harbor, N.Y.; Sambrook et al., (1989) Molecular Cloning: A Laboratory Manual. ^{2nd} ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. ^{4th} ed. Wiley-Liss; among others. The Current Protocols listed above are updated several times every year.

### Compositions

Disseminated metastatic disease is the primary cause of death among cancer patients. Cancer vaccination stimulates a systemic immune response against judiciously chosen tumor antigens expressed in the tumor cells that seeks out and destroys the disseminated tumor lesions. The development of effective cancer vaccines will require the identification of potent and broadly expressed tumor rejection antigens (TRAs) (Gilboa, E. Immunity 11, 263-270 (1999); Novellino, L., et al. Cancer Immunol. Immunother. 54, 187-207 (2005); Schietinger, A., et al. Semin. Immunol. 20, 276-285 (2008)) as well as effective adjuvants to stimulate a robust and durable immune response (Gilboa, E. Nature Rev. Cancer 4, 401-411 (2004); Melief, C. J. Immunity 29, 372-383 (2008); Pardoll, D. M. Nature Rev. Immunol. 2, 227-238 (2002)).

Alternative novel approaches to vaccination are provided herein. Embodiments of the invention comprise expressing new, and hence potent, antigens in tumor cells in situ. How to express new antigens in the disseminated tumor lesions, but not in normal tissue, have heretofore precluded the development of such strategies.

Nonsense mediated decay (NMD) prevents the expression of aberrant products in the cell. In preferred embodiments, inhibition of NMD in tumor cells leads to the expression of novel antigens and enhancement of the immunogenicity of tumor cells, leading to tumor rejection by the immune response. Delivery of siRNA targeted to NMD pathways in vivo can be used to inhibit NMD. However, non-targeted delivery of siRNA in vivo is not clinically practical because of cost consideration and anticipated toxicity. Targeting siRNA to the appropriate cells, tumor cells in this instance, would solve the problem.

The targeting strategy, described in embodiments herein, is a novel platform technology that can be used to develop improved methods of delivering therapeutic cargo such as small MW drugs, toxins, siRNAs, as well as therapeutic drugs, to cells in vivo.

In general, the invention provides compositions and methods for inhibiting nonsense-mediated mRNA decay (NMD) and/or a component of the NMD pathway in a cell. Embodiments of the invention are directed to a clinically useful approach to express new antigens in the disseminated tumor lesions of cancer patients by targeted inhibition of nonsense mediated decay (NMD) in the tumor cells. NMD is an evolutionary conserved mRNA surveillance pathway in eukaryotic cells that detects and eliminates mRNAs harboring premature termination codons (PTCs). Without wishing to be bound by theory, the central hypothesis is that upregulation of gene expression when NMD is inhibited in tumor cells will translate into therapeutically useful enhancement of tumor antigenicity, namely that the new products will function as effective tumor antigens, capable of eliciting an immune response which will contribute to tumor rejection Inhibition will be accomplished using any molecule which specifically inhibits NMD factors, for example, siRNAs, enzymes, peptides, proteins, oligonucleotides, organic or inorganic compounds, and the like which will be targeted to tumor cells or any other abnormal cell by conjugation to ligands expressed by cells of the tumor stroma. To target the bispecific or multi-specific agents embodied by the invention to the tumors in vivo, an antisense molecule specific for nonsense mediated decay which modulate expression and function of one or more molecules associated with nonsense mediated decay pathways, is conjugated, linked, fused and the like, to a second molecule which binds to the cell surface molecules of the normal cell constituents of the tumor stroma, referred to herein as “stromal cell(s).” Normal cell constituents of the tumor stroma comprise for example, cells such as macrophages, dendritic cells, endothelial cells, fibroblasts and the like. The ligand or molecule which binds to stromal cell molecules comprises integrin binding molecules, such as for example, peptides having an RGD motif; ligands that bind to endothelial specific molecules such as for example, vascular endothelial growth factor receptor-1 (VEGFR-1), vascular endothelial growth factor receptor (VEGFR-2), Tie2; peptides comprising a C-terminal arginine (CendR) motif or C-terminal lysine motif (which bind to neurophilin and integrin), fibronectin, vitronectin, collagen, laminin, any peptides; fibroblast antigens, such as for example, fibroblast activation protein (FAP), ligands which target glucose-regulated protein 78 (GRP78), peptides binding to integrins, Pie42 peptides, and the like. One of skill in the art will appreciate that any other antigen or protein associated with vascular or other tumor-associated stromal cells can be a target for the immunogenic compositions, including those that are presently known and those yet to be identified.

In preferred embodiments, the bispecific or multi-specific NMD targeted agents comprise one or more ligands for binding to integrins. In all embodiments, the first domain comprising the targeting molecule is any type of molecule which can specifically bind or target the NMD composition to the desired target in vivo or in vitro. Examples include, aptamers, antibodies, antibody fragments, synthetic molecules, small molecules and the like.

In some embodiments, the aptamer is a multimer. “Multimer” is used herein, for purposes of the specification and claims, to mean two or more aptamers are linked together. Thus, a multimer may comprise a dimer, trimer, tetramer, etc. The aptamer molecules in a multimer may be of the same nucleic acid sequence and/or binding specificity, as compared to other aptamer molecules present as part of the multimer.

Generation of Aptamers:

Aptamers are high affinity single-stranded nucleic acid ligands which can be isolated from combinatorial libraries through an iterative process of in vitro selection known as SELEX™ (Systemic Evolution of Ligands by EXponential enrichment). Aptamers exhibit specificity and avidity comparable to or exceeding that of antibodies, and can be generated against most targets. Unlike antibodies, aptamers, or in this instance aptamer-siRNA fusions, can be synthesized in a chemical process and hence offer significant advantages in terms of reduced production cost and much simpler regulatory approval process. Also, aptamers-siRNAs are not expected to exhibit significant immunogenicity in vivo.

In preferred embodiments, the siRNA is linked to at least one aptamer which is specific for a desired cell and target molecule. In other embodiments, the RNAi's are combined with two aptamers. The various permutations and combinations for combining aptamers and RNAi's is limited only by the imagination of the user.

Methods of the present disclosure do not require a priori knowledge of the nucleotide sequence of every possible gene variant (including mRNA splice variants) targeted by the RNAi or analog thereof.

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

In a preferred embodiment, the first domain comprising the targeting molecule is an aptamer. Preferably, the targeting domain is specific for targets in the stroma. In preferred embodiments, aptamers specifically target, for example, siRNA, to a desired nucleic acid target. Aptamers are oligonucleotide-based ligands that exhibit specificity and avidity comparable or superior to antibodies. However, unlike antibodies, aptamers are synthesized chemically in cell free system, and offer a more straightforward and cost effective manufacturing process and a vastly simpler regulatory approval process for clinical use.

Other examples of ligands used as a targeting molecule in the first domain are provided below.

Stromal Cell Receptor/Ligands:

Integrins are one type of stromal cell surface molecules and are the targets of choice in preferred embodiments. The integrins are a family of αβ heterodimeric receptors that mediate dynamic linkages between extracellular adhesion molecules and the intracellular actin cytoskeleton. Integrins are expressed by all multicellular animals. In mammals, 18 α-subunit genes and eight n-subunit genes encode polypeptides that combine to form 24 different receptors. Both integrin subunits are noncovalently associated, type I transmembrane proteins with large extracellular domains and short cytoplasmic domains of 700-1100 and 30-50 residues, respectively. Other peptides which specifically bind to tumor vasculature can be identified by any means, such as for example, phage display, high-throughput screening assays, etc.

Integrins differ from other cell-surface receptors in that they bind their ligands with a low affinity (10⁶-10⁹ liters/mole) and that they are usually present at 10-100 fold higher concentration on the cell surface. Integrin-ligand interactions are accompanied by clustering and activation of the integrins on the cell surface, which is also accompanied by the transduction of signals into intracellular signal transduction pathways that mediate a number of intracellular events. Signaling through integrins depends on the formation of focal adhesions, dynamic sites in which cytoskeletal and other proteins are concentrated and which regulate migration and the shape of a cell.

A number of integrins mediate the binding of a cell to the C-terminal globular domain of laminin. α₆β₁ has been shown to associate with proteins of the C-terminal globular domain of the laminin A1 chain. A₃β₁ has also been demonstrated to bind the globular domain of the laminin A chain. A7β₁ binds specifically to laminin.

Integrins can adhere (bind) an array of ligands. Common ligands are for example fibronectin and laminin, which are both part of the extracellular matrix or basal lamina's. Both of the ligands mentioned above are recognized by multiple integrins. For adhesion to ligands both integrin subunits are needed, as is the presence of cations. The alpha chain is the part that has cation binding sites.

Other examples comprise: stromal derived factor 1 (SDF-1), stromal derived factor 2 (SDF-2), MCP-1, MIP-1α, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, neurophilin, MAdCAM-1, neuropilin-1, α4 integrins, α5 integrins, beta defensins 3 and 4.

In a preferred embodiment, the targeting domain is an aptamer.

In a preferred embodiment, the ligand (cell targeting or binding domain) specifically binds to receptors on cells in the tumor stroma or tumor vasculature. Thus, for example, the ligand or molecule which binds to stromal cell molecules comprises integrin binding molecules, such as for example, peptides having an RGD motif; ligands that bind to endothelial specific molecules such as for example, vascular endothelial growth factor (VEGF), vascular endothelial growth factor receptor (VEGFR-2), Tie2; fibroblast antigens, such as for example, fibroblast activation protein (FAP), aptamers, and the like. One of skill in the art will appreciate that any other antigen or protein associated with vascular or other tumor-associated stromal cells can be a target for the immunogenic compositions, including those that are presently known and those yet to be identified.

In another preferred embodiment, the NMD targeted agents comprise a targeting domain which specifically binds to any one or more stromal ligands. Examples include, without limitation: SDF-1 (stromal cell-derived factor-1; designated Chemokine (C-X-C motif) ligand 12 (CXCL12)), Galectins (e.g. Galectin-1), fibroblast-activation protein (FAP), endoglyx-1, endosialin, CD1.8, Cadherins (e.g. Cadherin-11), Calretinin, CD10, CD117, Desmin, Endosialin, Fibroblast-Activation Protein (FAP), Laminin gamma2 chain, Neural Ganglioside GD2, Nucleostemin, Snep (stromal nidogen extracellular matrix protein), Tenascin, CD13, CD29, CD44, CD45, CD63, CD73, CD90, CD105, CD166, STRO-1, HOP-26 (CD63), CD49a and SB-10 (CD166), Alpha and beta subunits of inhibin/activin, Alpha-smooth muscle actin and other stromal markers in endometrial mucosa.

In another preferred embodiments, the NMD agents comprise targeting molecules which specifically bind to glucose-regulated protein 78 (GRP78). Examples include ligand peptides (linear or cyclic) comprising: WIFPWIQL (SEQ ID NO: 1), WDLAWMFRLPVG (SEQ ID NO: 2), CTVALPGGYVRVC (SEQ ID NO: 3), GIRLRG (SEQ ID NO: 4), iRGD (CRGDK/RGPD/EC (K. Sugahara et al, Cancer Cell 16, pp 510-520 (2009)), R/KXXR/K (SEQ ID NO: 5), variants, mutants or analogs thereof.

In another preferred embodiment, the targeting peptide comprises a CX_{N+1}CX_{N+1}CX_{N+1}C motif, wherein X is a variable amino acid and n=0 to 50.

In another preferred embodiment, the targeting peptide comprises a RGD motif.

In all of the peptides, the amino acids can be replaced in one or more positions with analogs, variants and the like. The peptides may also possess cyclic structures, isosteres, enantiomers etc.

In another preferred embodiment, the agents embodied herein optionally comprise cell-penetrating domains allowing for the enhanced intra-cellular delivery.

In a preferred embodiment, a composition for inducing novel antigens in abnormal cells comprises a multi-domain molecule having at least one target specific domain and at least one domain, which modulates expression and function of molecules associated with nonsense mediated decay pathways. As used herein, “multi” refers to at least two.

In another preferred embodiment, the multi-domain molecule comprises at least one target specific domain and at least two domains which modulate expression and function of one or more molecules associated with nonsense mediated decay pathways. Examples of such molecules comprise: RENT1, RENT2, eIF4A, UPF1, UPF2, UPF3B, RNPS1, Y14, MAGOH, NMD1, SMG, or combinations thereof.

In another preferred embodiment, the multi-domain molecule comprises at least two target specific domains and at least one domain which modulates expression and function of one or more molecules associated with nonsense mediated decay pathways.

In another preferred embodiment, the target specific domains comprise specificities for similar target molecules, different target molecules, or combinations thereof.

In another preferred embodiment, the domains which modulate expression and function of one or more molecules associated with nonsense mediated decay pathways modulate the expression and function of similar targeted molecules, different targeted molecules or combinations thereof.

In another preferred embodiment, the target specific domains are specific for target cell molecules, the target cell comprising: cells of the tumor stroma, an infected cell, a tissue specific cell, an adipocyte, a stem cell, an immune cell, an organ specific cell or a transformed cell.

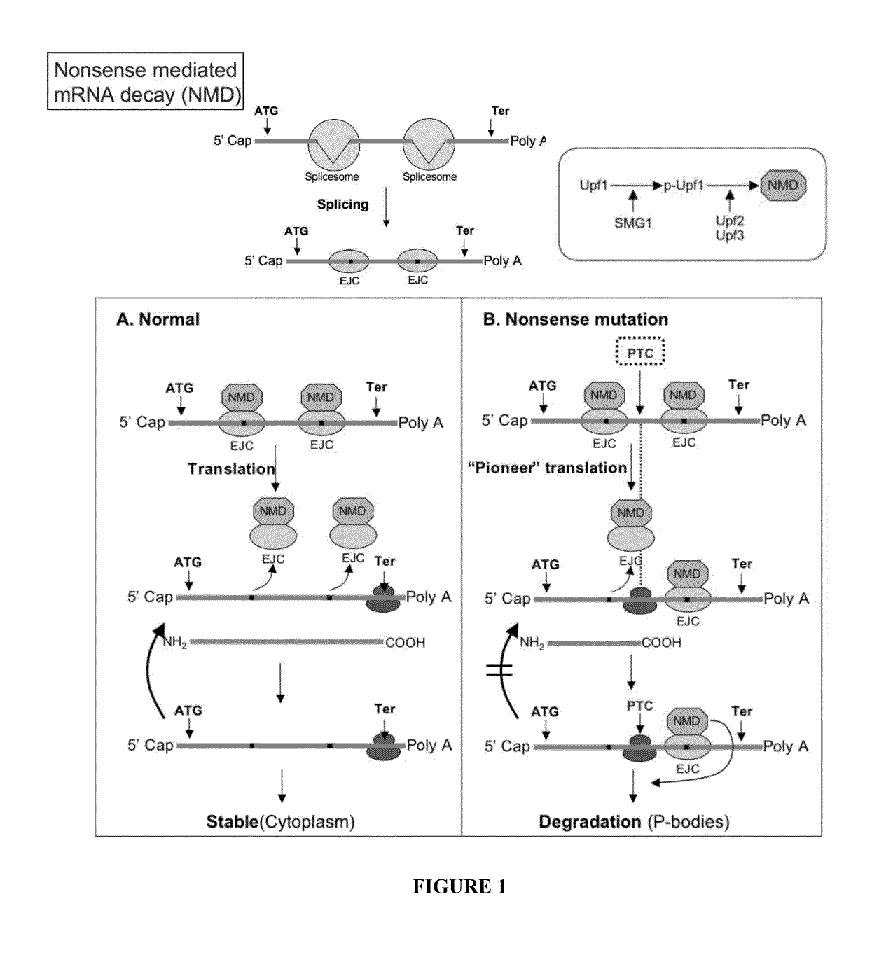

Nonsense mediated (mRNA) decay (NMD): FIG. 1 is a schematic representation showing the mechanism by which the NMD process prevents the accumulation of PTC containing mRNAs in eukaryotic cells. In brief, removal of introns from the pre-mRNA leaves behind an exon junction complex (EJC) demarcating the splice junctions (Panel A). An NMD complex consisting of several factors including Upf1, Upf2 and Up3 is then assembled on each EJC as shown in Panel B. SMG1, which phosphorylates Upf1, and Upf1 are the two key rate limiting factors in the formation of the complex. When the mRNA undergoes the first round of translation, called the “pioneer translation”, the EJC/NMD complex is removed, presumably as a result of the translational machinery moving through the region, thereby rendering the mRNA stable and competent for additional rounds of translation. If a PTC is present in an exon (other than the last exon), for example as a result of an inframe nonsense mutation, the EJC/NMD complexes downstream to the PTC are not removed from the mRNA. The attached NMD complex then triggers the degradation of the mRNA.

In preferred embodiments, a composition comprising oligonucleotides directed against NMD-specific factors inhibit NMD in tumor cells (see, for example, FIG. 1). The siRNAs targeted to the tumor stroma by conjugation to peptide or oligonucleotide-based ligands. Targeting the siRNAs to tumor cells is a preferred method for the therapeutic use of this approach since upregulation of new products in nontransformed cells could expose normal tissue to immune destruction creating an “autoimmune inferno”.

NMD-mediated degradation of mRNA is not limited to instances of mutations or recombinations generating PTCs. Error-free mRNAs containing short 5′ open reading frames (ORFs), mRNAs which are regulated by stop codon read-through, leaky scanning for translation initiation, or regulated frameshifting or mRNA can be also recognized by NMD.

Physiological Roles of NMD:

It was initially thought that the main role of NMD was to maintain the proteome integrity of the cell by eliminating transcripts with nonsense mutations generating PTCs yielding truncated products. Indeed, over 30% of genetic disorders are caused by PTC. In several instances the severity of the disease, e.g. β-thalassemia, correlates with the NMD-controlled degradation of the mutant mRNA. Yet, nonsense mutations generating PTCs are rare events and it is unlikely that the NMD system has evolved to counter their potential deleterious effects. There is in fact accumulating evidence that the main and physiological role of the NMD is to regulate normal gene expression.

An important role of NMD is to maintain splicing integrity. The efficiency and accuracy of splicing is notoriously imperfect. Such transcripts will often contain PTCs and hence become targets for NMD elimination. NMD is also responsible for the elimination of transcripts encoding nonproductively rearranged T cell receptors and immunoglobulin chains. A significant proportion of gene products (>15%) that are upregulated when NMD is inhibited, such as by targeting Upf1 with siRNA are involved in amino acid biosynthesis and transcription factors which coordinate cellular responses to starvation. Since starvation also downregulates translation thru phosphorylation and inhibition of eIF2a, which in turn inhibits NMD efficiency, it appears that the response to starvation is in part under NMD control. NMD is also implicated in several instances of products autoregulating alternative splicing (e.g., serine-arginine (SR)-rich proteins and hnRNP splicing factors such as SC35, calpain, CDC-like kinases, biosynthesis of selenoproteins, and telomere synthesis.

Other functions of Upf1 and SMG1. In preferred embodiments, a method of inducing or enhancing tumor antigenicity comprises a composition having a ligand specific for a target cell conjugated to an agent which inhibits NMD in tumor cells, for example, siRNAs specific for key NMD factors. Upf1 also promotes the replication-dependent decay of histone mRNA which is required for cell cycle progression. SMG1 is a kinase which also phosphorylates and inactivates p53; siRNA inhibition of SMG-1 in U2OS cells results in the accumulation of dsDNA break and activation of ATM- or ATR-mediated checkpoint responses. Both Upf1 and SMG1 were implicated in telomere maintenance by facilitating the binding of telomere repeat-containing RNA (TERRA) to telomeres.

Cancer cells accumulate elevated level of PTC containing NMD mRNA substrates. About 15% of cancers exhibit defects in DNA mismatch repair (MMR) often manifested as mirosatellite instability (MSI). Such defects affecting many products, including products associated with tumor progression such as TGFβRII, APAF-1, IGFIIR, BAX, PTEN, RHAMM, give rise to frameshift mutation ending in PTCs. Such PTC-containing transcripts are under NMD control whereby Upf1 siRNA mediated inhibition of NMD in a human colorectal cancer cell line exhibiting an MSI phenotype stabilized the frameshifted mutant transcripts. Such products could provide a source of tumor-specific antigenic determinants downstream the recombination site. Thus, increased immune infiltrate are seen in tumors with MIS phenotype would correlate with the levels of Upf1 in the tumors. Inhibiting NMD further augments the production of such tumor-specific antigens. In a preferred embodiment, the gene silencing agent (the RNAi) is targeted to the appropriate cells in vivo using molecules which bind to cells present in the tumor stroma, for example, peptides comprising an RGD motifs, integrins, and the like. Targeting of polynucleotides, without limitation, any one or more components of a nonsense mediated decay pathway. The display of new antigens or an increase in antigens that can be recognized by the immune system as abnormal or foreign results in the destruction of that cell.

In another preferred embodiment, a composition comprising a targeting agent and a gene silencing agent down-regulate or abrogate nonsense mediated decay pathways. In a preferred embodiment, the gene silencing agent is an RNAi (siRNA/shRNA). However, the silencing agent which is specific for nonsense mediated pathways or factors associated with such pathways can be any type of molecule such as, for example, peptides, proteins, oligonucleotides, organic compounds, inorganic compounds and the like.

Other Target Molecules:

The target molecule can be, for example, an extracellular domain of a growth factor receptor. The ligand that would be selected in the cell binding domain of the molecule would be one which bound to a desired cell surface molecule such as for example, the VEGF receptor. Exemplary receptors to which the ligands would bind to include the c-erbB-2 protein product of the HER2/neu oncogene, epidermal growth factor (EGF) receptor, basic fibroblast growth receptor (basic FGF) receptor and vascular endothelial growth factor receptor, E-, L- and P-selectin receptors, folate receptor, CD4 receptor, CD19 receptor, a, β-integrin receptors and chemokine receptors.

In another preferred embodiment, a ligand for use in the cell binding domain comprise any peptide identified by phage display libraries, high throughput screening assays or any other means at the disposal of a user both current and discovered in the future. The ligands would be in preferred embodiments, specific for tumor stroma or tumor vasculature.

In other preferred embodiments, the cell binding domain may also be conjugated to transporter proteins to increase the transportation of the molecules specific for NMD factors across membranes e.g. blood brain barrier, intestines, etc.

A “chemotherapeutic agent” which can also be the cargo moiety for treatment of a tumor is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, kcoxifene, LY117018, onapristone, and toremifene (Farcston); and anti-androgens such as flutamide, nilutamide, bicalutamide, leuprolide, and goserelin; and pharmaceutically acceptable salts, acids or derivatives of any of the above.

The cargo moiety delivered in association with the molecules embodied herein, may be any of various therapeutic and diagnostic agents which are desired to be delivered to a target. Therapeutic agents which can be included as cargo moieties in the delivery system of the present invention illustratively include but are not limited to therapeutic compounds such as an analgesic, an anesthetic, an antibiotic, an anticonvulsant, an antidepressant, an antimicrobial, an anti-inflammatory, anti-migraine, an antineoplastic, an antiparasitic, an antitumor agent, an antiviral, an anxiolytic, a cytostatic, cytokine, a hypnotic, a metastasis inhibitor, a sedative and a tranquilizer.

In another preferred embodiment, the molecules are labeled with a detectable agent, which are administered to a patient for the in vivo imaging of a tumor. The specific delivery of the detectable agent provides a vastly superior means of specific detection of a tumor or desired target cell and decreases any background noise, allowing for the early detection and diagnosis of, for example, a tumor.

Immune Cell Co-Stimulation:

In some embodiments, the compositions are administered with one or more immune cell co-stimulatory agents. These agents comprise aptamers with specificity for an immune cell stimulatory molecule. Embodiments comprise a monospecific, bispecific or multi-specific costimulatory agent which delivers an immune cell co-stimulatory signal, activating immune cells. Co-stimulation of immune cells is mediated by ligands which interact with receptors on the surface of the immune cells, e.g. CD28, 4-1BB, OX40, etc. Tumor cells do not express costimulatory ligands and hence presentation of tumor antigens by the tumor cells does not potentiate the naturally occurring or a vaccine-induced antitumor immune response. The provision of such costimulatory products to tumor cells or tumor stroma enhances antitumor immunity and can lead to tumor regression.

To target the monospecific (e.g. directed to the same antigen or directed to the same antigen but to different epitopes), bispecific or multi-specific costimulatory agents embodied by the invention to the tumors in vivo, a molecule specific for costimulatory ligand is conjugated, linked, fused and the like, to a second molecule which binds to the cell surface molecules of the normal cell constituents of the tumor stroma, referred to herein as “stromal cell(s).” Normal cell constituents of the tumor stroma comprise for example, cells such as macrophages, dendritic cells, endothelial cells, fibroblasts and the like. The bispecific or multi-specific costimulatory agent specific for an immune cell co-stimulatory molecule can be for example, an aptamer, antibody, peptide and the like. The ligand or molecule which binds to stromal cell molecules comprises integrin binding molecules, such as for example, peptides having an RGD motif; ligands that bind to endothelial specific molecules such as for example, vascular endothelial growth factor (VEGF), vascular endothelial growth factor receptor (VEGFR-2), Tie2; fibroblast antigens, such as for example, fibroblast activation protein (FAP), and the like. One of skill in the art will appreciate that any other antigen or protein associated with vascular or other tumor-associated stromal cells can be a target for the immunogenic compositions, including those that are presently known and those yet to be identified.

Without wishing to be bound by theory, co-stimulation of immune cells specific for molecules expressed on underlying non-transformed stromal cells (including neovasculature as well as interstitial tissue, for example) can also improve the effectiveness of the disclosed methods and compositions in limiting tumor growth and promoting cancer regression by other mechanisms. Stroma encompasses neovasculature as well as fibroblasts, and in general, all non-transformed cells within a tumor microenvironment. For example, immune mediated responses to desired target cells (via cytotoxic T lymphocytes (CTLs)) or antibody dependent cytotoxic cells (ADCC) can result in neovasculature permeabilization and initiation of inflammatory events that result in recruitment and translocation of immune effectors, such as CTLs, targeting the neoplastic cells within primary tumor and metastatic lesions. Moreover, as attacks based, for example, on T cell recognition of endothelial cell MHC-peptide complexes occur in the luminal environment, any immune suppressive influence of the tumoral environment is minimized.

Another advantage is that the stromal cell molecules can be considered “universal” antigens. Thus, any tumor cells which evade the immune system via various mechanisms are still targeted as the therapeutic bispecific or multi-specific costimulatory agents are targeting stromal cells, and their antigens, and not the tumor cells, and their antigens.

In a preferred embodiment, a bispecific or multi-specific costimulatory agent comprises a domain which is specific for an immune cell co-stimulatory molecule, for example, a 4-1BB and second domain specific for products expressed on the surface of normal cells which make up the tumor stroma. An example of targeting ligand would be a molecule with an RGD motif which will bind to integrin unregulated on tumor vasculature. In other embodiments, the bispecific or multi-specific costimulatory agent comprises combinations of one or more domains binding to co-stimulatory molecules and one or more domains which bind to cells in the tumor stroma. 4-1 BB is a major costimulatory receptor expressed on CD8⁺ T cells and peptides with RGD motifs bind to integrins. Integrins are unregulated on tumor endothelial cells, and RGD ligation does not lead to any great degree of internalization making it suitable for use as one of the domains in the therapeutic bispecific or multi-specific costimulatory agents of the invention. The 4-1BB-RGD bound to cells expressing integrins in vitro, in effect “coating” the tumor vascular cells with the 4-1BB ligand. These co-stimulatory signals activate the immune response and the immune cells target the cells in the tumor stroma.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, antigen presenting cells and derivatives, precursors or progenitors of the above cell types.

“Immune effector cells” refers to cells, and subsets thereof, e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10-(IL-10-)secreting T regulatory type 1 (Tr1) cells, transforming growth factor-β-(TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

Numerous costimulatory molecules have been identified playing a role in the initiation of immune responses by T and B lymphocytes. Signals provided through CD28-B7 interactions are essential for initial naïve T cell activation leading to increased IL-2 production and IL-2Rα (CD25) expression. NKG2D binds to the MHC-related proteins MIC and Rae-1 and induces IL-2 production and proliferation. In other cell types, such as B cells, activation requires CD40-CD40L interactions for proper antibody response: promoting survival, cytokine receptor expression, and inducing antibody class switch. In addition to the costimulatory pathways that are important in naïve lymphocyte activation, other costimulatory molecules play a role in effector/memory lymphocyte activation.

The costimulatory receptors ICOS, OX-40, 4-1BB, and CD27 bind to their ligands B7h, OX-40L, 4-1BBL, and CD70, respectively, to enhance the activation, survival, and cytokine secretion of effector/memory, but not naïve T and B cells. These costimulatory receptors and their ligands are not constitutively expressed but are induced on differentiated T cells, and their ligands are not restricted to APCs. T cell activation generally incorporates a self-limiting mechanism, such as inhibitory costimulators, to regulate T cell tolerance and attenuate the immune response. The expanding set of inhibitory costimulators currently includes CTLA-4 (CD152), PD-1, and BTLA. While expression of these molecules is induced following T cell activation, they are absent on naïve T cells. Lastly, B7-H3 is a new costimulatory ligand originally described to induce T cell proliferation and IFN-γ production through an as of yet unidentified receptor.

In preferred embodiments, the immune cell co-stimulatory molecules induce an immune response. In a preferred embodiment, a co-stimulatory domain of the therapeutic bispecific or multi-specific costimulatory agents herein comprise one or more molecules: 4-1BB (CD137), OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-13, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

Examples of immune cells comprise T cells (T lymphocytes), B cells (B lymphocytes), antigen presenting cells, dendritic cells, monocytes, macrophages, myeloid suppressor cells, natural killer (NK) cells, NKT cells, NKT suppressor cells, T regulatory cells (Tregs), T suppressor cells, cytotoxic T lymphocytes (CTLs), CTL lines, CTL clones, CTLs from tumor, inflammatory, or other infiltrates and subsets thereof.

Natural killer T (NKT) cells are a heterogeneous group of T cells that share properties of both T cells and natural killer (NK) cells. Many of these cells recognize the non-polymorphic CD1 d molecule, an antigen-presenting molecule that binds self- and foreign lipids and glycolipids. NKT cells are a subset of T cells that co-express an αβ T cell receptor (TCR), but also express a variety of molecular markers that are typically associated with NK cells, such as NK1.1. They differ from conventional αβ T cells in that their TCRs are far more limited in diversity and in that they recognize lipids and glycolipids presented by CD1 d molecules, a member of the CD1 family of antigen presenting molecules, rather than peptide-MHC complexes. NKT cells include both NK1.1⁺ and NK1.1⁻, as well as CD4⁺, CD4⁻, CD8⁺ and CD8⁻ cells. Natural Killer T cells share other features with NK cells as well, such as CD16 and CD56 expression and granzyme production. NKT cells are classified into type I (invariant) and type II (non-invariant) cells in mice and humans. The best known subset of CD1d-dependent NKT cells expresses an invariant T cell receptor α (TCR-α) chain. These are referred to as type I or invariant NKT cells (iNKT) cells.

In a preferred embodiment, the compositions of the present invention are targeted to immune cell co-stimulatory molecules, for example, 4-1BB, CD27 (CD27 ligand is CD70), HVEM, LTβ receptors or ligands thereof.

### Generation of Interference RNA

Detailed methods of producing the RNAi's are described in the examples section which follows. The RNAi's of the invention can also be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, the RNAi's of the invention are chemically synthesized using appropriately protected ribonucleotide phosphoramidites and a conventional DNA/RNA synthesizer. The RNAi can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, RNAi can also be expressed from recombinant circular or linear DNA plasmids using any suitable promoter. Suitable promoters for expressing RNAi of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the RNAi in a particular tissue or in a particular intracellular environment. RNAi's of the invention can be expressed from a recombinant plasmid either as two separate, complementary RNA molecules, or as a single RNA molecule with two complementary regions.

Selection of plasmids suitable for expressing RNAi of the invention, methods for inserting nucleic acid sequences for expressing the RNAi into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16:948-958; Lee N S et al, (2002), Nat. Biotechnol. 20: 500-505; and Paul C P et al. (2002), Nat. Biotechnol. 20: 505-508, the entire disclosures of which are herein incorporated by reference.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or anti sense strands are located 3′ of the promoter, so that the promoter can initiate transcription of the sense or antisense coding sequences.

Any viral vector capable of accepting the coding sequences for the siRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the RNAi into the vector, and methods of delivering the viral vector to the cells of interest are within the skill in the art. See, for example, Dornburg R (1995), Gene Therap. 2: 301-310; Eglitis M A (1998), Biotechniques 6: 608-614; Miller A D (1990), Hum Gene Therap. 1: 5-14; and Anderson W F (1998), Nature 392: 25-30, the entire disclosures of which are herein incorporated by reference.

A suitable AV vector for expressing the RNAi's of the invention, a method for constructing the recombinant AV vector, and a method for delivering the vector into target cells, are described in Xia H et al. (2002), Nat. Biotech. 20: 1006-1010. Suitable AAV vectors for expressing the RNAi's of the invention, methods for constructing the recombinant AAV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosure of which are herein incorporated by reference.

The ability of an RNAi containing a given target sequence to cause RNAi-mediated degradation of the target mRNA can be evaluated using standard techniques for measuring the levels of RNA or protein in cells. For example, RNA of the invention can be delivered to cultured cells, and the levels of target mRNA can be measured by Northern blot or dot blotting techniques, or by quantitative RT-PCR. RNAi-mediated degradation of target mRNA by an siRNA containing a given target sequence can also be evaluated with animal models, such as mouse models. RNAi-mediated degradation of the target mRNA can be detected by measuring levels of the target mRNA or protein in the cells of a subject, using standard techniques for isolating and quantifying mRNA or protein as described above.

In a preferred embodiment, siRNA molecules target overlapping regions of a desired sense/antisense locus, thereby modulating both the sense and antisense transcripts. In another preferred embodiment, a composition comprises siRNA molecules, of either one or more, and/or, combinations of siRNAs, siRNAs that overlap a desired target locus, and/or target both sense and antisense (overlapping or otherwise). These molecules can be directed to any target that is desired for potential therapy of any disease or abnormality. Theoretically there is no limit as to which molecule is to be targeted. Furthermore, the technologies taught herein allow for tailoring therapies to each individual.

In preferred embodiments, the oligonucleotides can be tailored to individual therapy, for example, these oligonucleotides can be sequence specific for allelic variants in individuals, the up-regulation or inhibition of a target can be manipulated in varying degrees, such as for example, 10%, 20%, 40%, 100% expression relative to the control. That is, in some patients it may be effective to increase or decrease target gene expression by 10% versus 80% in another patient.

Up-regulation or inhibition of gene expression may be quantified by measuring either the endogenous target RNA or the protein produced by translation of the target RNA. Techniques for quantifying RNA and proteins are well known to one of ordinary skill in the art. In certain preferred embodiments, gene expression is inhibited by at least 10%, preferably by at least 33%, more preferably by at least 50%, and yet more preferably by at least 80%. In particularly preferred embodiments, of the invention gene expression is inhibited by at least 90%, more preferably by at least 95%, or by at least 99% up to 100% within cells in the organism. In certain preferred embodiments, gene expression is up-regulated by at least 10%, preferably by at least 33%, more preferably by at least 50%, and yet more preferably by at least 80%. In particularly preferred embodiments, of the invention gene expression is up-regulated by at least 90%, more preferably by at least 95%, or by at least 99% up to 100% within cells in the organism.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

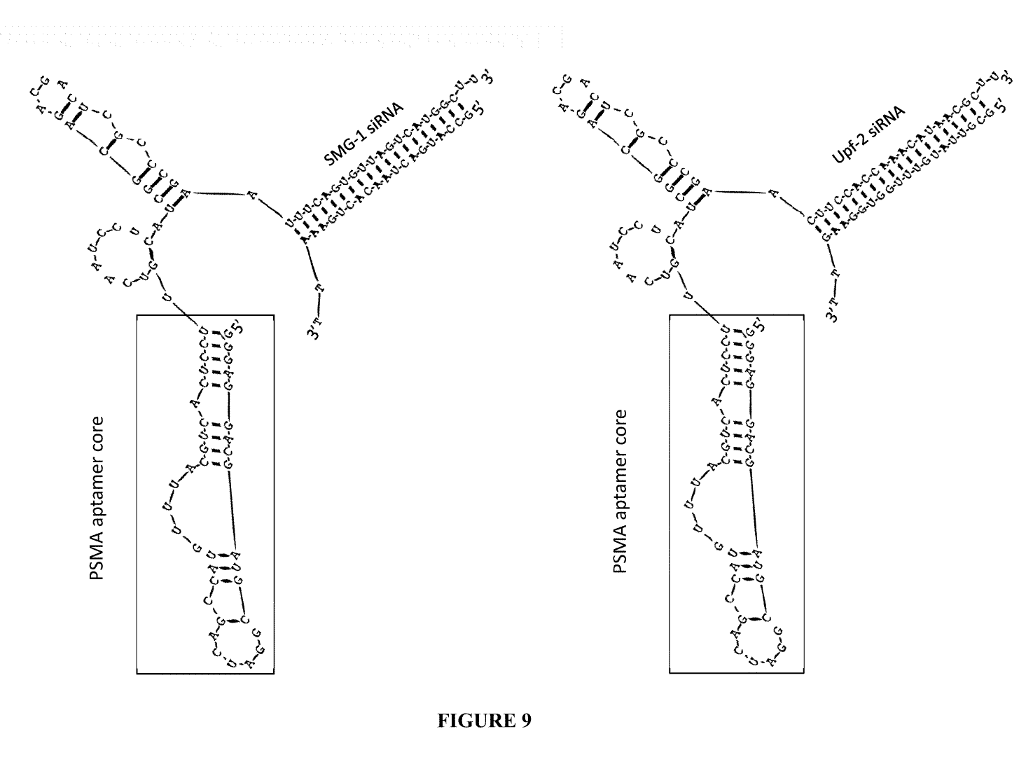

In a preferred embodiment, small interfering RNA (siRNA) either as RNA itself or as DNA, is delivered to a cell using aptamers. FIG. 9 provides a schematic illustration of aptamer targeted siRNAs. Many different permutations and combinations of aptamers and RNAi's can be used. For example, the siRNA can be attached to one or more aptamers or encoded as a single molecule so that the 5′ to 3′ would encode for an aptamer, the siRNA and an aptamer. These can also be attached via linker molecules. The composition can also comprise in a 5′ to 3′ direction an aptamer attached to another aptamer via a linker which are then attached to the siRNA. These molecules can also be encoded in the same combination. Compositions can include various permutations and combinations. The composition can include siRNAs specific for different polynucleotide targets.

In certain embodiments, the nucleic acid molecules of the present disclosure can be synthesized separately and joined together post-synthetically, for example, by ligation (Moore et al., Science 256:9923, 1992; Draper et al., PCT Publication No. WO 93/23569; Shabarova et al., Nucleic Acids Res. 19:4247, 1991; Bellon et al., Nucleosides & Nucleotides 16:951, 1997; Bellon et al., Bioconjugate Chem. 8:204, 1997), or by hybridization following synthesis or deprotection.

In further embodiments, RNAi's can be made as single or multiple transcription products expressed by a polynucleotide vector encoding one or more siRNAs and directing their expression within host cells. An RNAi or analog thereof of this disclosure may be further comprised of a nucleotide, non-nucleotide, or mixed nucleotide/non-nucleotide linker that joins the aptamers and RNAi's. In one embodiment, a nucleotide linker can be a linker of more than about 2 nucleotides length up to about 50 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In accordance with the invention, siRNA oligonucleotide therapies comprise administered siRNA oligonucleotide which contacts (interacts with) the targeted mRNA from the gene, whereby expression of the gene is modulated. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an siRNA oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the siRNA oligonucleotide.

In another preferred embodiment, the nucleobases in the siRNA may be modified to provided higher specificity and affinity for a target mRNA. For example nucleobases may be substituted with LNA monomers, which can be in contiguous stretches or in different positions. The modified siRNA, preferably has a higher association constant (Kₐ) for the target sequences than the complementary sequence. Binding of the modified or non-modified siRNA's to target sequences can be determined in vitro under a variety of stringency conditions using hybridization assays and as described in the examples which follow.

Aptamer-siRNA oligonucleotides may be used in combinations. For instance, a cocktail of several different siRNA modified and/or unmodified oligonucleotides, directed against different regions of the same gene, may be administered simultaneously or separately.

In the practice of the present invention, target gene products may be single-stranded or double-stranded DNA or RNA. Short dsRNA can be used to block transcription if they are of the same sequence as the start site for transcription of a particular gene. See, for example, Janowski et al. Nature Chemical Biology, 2005, 10:1038. It is understood that the target to which the siRNA oligonucleotides of the invention are directed include allelic forms of the targeted gene and the corresponding mRNAs including splice variants. There is substantial guidance in the literature for selecting particular sequences for siRNA oligonucleotides given a knowledge of the sequence of the target polynucleotide. Preferred mRNA targets include the 5′ cap site, tRNA primer binding site, the initiation codon site, the mRNA donor splice site, and the mRNA acceptor splice site.

Where the target polynucleotide comprises a mRNA transcript, sequence complementary oligonucleotides can hybridize to any desired portion of the transcript. Such oligonucleotides are, in principle, effective for inhibiting translation, and capable of inducing the effects described herein. It is hypothesized that translation is most effectively inhibited by the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

Chimeric/Modified RNAi's:

In accordance with this invention, persons of ordinary skill in the art will understand that mRNA includes not only the coding region which carries the information to encode a protein using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. In preferred embodiments, the oligonucleotide is targeted to a translation initiation site (AUG codon) or sequences in the coding region, 5′ untranslated region or 3′-untranslated region of an mRNA. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

### Pharmaceutical Compositions

The invention also includes pharmaceutical compositions containing a bispecific or multi-specific binding agent. In some embodiments, the compositions are suitable for internal use and include an effective amount of a pharmacologically active conjugate of the invention, alone or in combination, with one or more pharmaceutically acceptable carriers. These conjugated bispecific or multi-specific costimulatory agents are especially useful in that they have very low, if any toxicity.

The patient having a pathology, e.g. the patient treated by the methods of this invention can be a mammal, or more particularly, a human. In practice, the agents, are administered in amounts which will be sufficient to exert their desired biological activity.

The pharmaceutical compositions of the invention may contain, for example, more than one specificity. In some examples, a pharmaceutical composition of the invention, containing one or more compounds of the invention, is administered in combination with another useful composition such as an anti-inflammatory agent, an immunostimulator, a chemotherapeutic agent, an antiviral agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of a bispecific or multi-specific costimulatory agent composition and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic bispecific or multi-specific costimulatory agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. Combination therapy also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

For any agent used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the peptides described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Dosage amount and interval may be adjusted individually to provide plasma levels of the active moiety which are sufficient to maintain therapeutic effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferable between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or other known methods.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compositions of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compositions for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, suppositories, ointments, lotions, aerosol sprays and gels, wherein the concentration of active ingredient would typically range from 0.01% to 15%, w/w or w/v.

The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate. The dosage regimen utilizing the molecules is selected in accordance with a variety of factors including type, species, age, weight, sex and medical condition of the patient; the severity of the condition to be treated; the route of administration; the renal and hepatic function of the patient; and the particular molecule or salt thereof employed. An ordinarily skilled physician or veterinarian can readily determine and prescribe the effective amount of the drug required to prevent, counter or arrest the progress of the condition. Compounds of the present invention may be administered in a single daily dose, or the total daily dosage may be administered in divided doses of two, three or four times daily.

The invention has been described in detail with reference to preferred embodiments thereof. However, it will be appreciated that those skilled in the art, upon consideration of this disclosure, may make modifications and improvements within the spirit and scope of the invention. Embodiments of the invention may be practiced without the theoretical aspects presented. Moreover, the theoretical aspects are presented with the understanding that Applicants do not seek to be bound by the theory presented.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

While various embodiments of the present invention have been described above, it should be understood that they have been presented by way of example only, and not limitation. The following non-limiting examples are illustrative of the invention.

### Example 1

### Induction of Tumor Immunity by Targeted Inhibition of Nonsense-Mediated mRNA Decay

Disseminated metastatic disease is the primary cause of death among cancer patients. Cancer vaccination stimulates a systemic immune response against judiciously chosen tumor antigens expressed in the tumor cells that seeks out and destroys the disseminated tumor lesions. The development of effective cancer vaccines will require the identification of potent and broadly expressed TRAs as well as effective adjuvants to stimulate a robust and durable immune response. An alternative approach to vaccination is to express new, and hence potent, antigens in tumor cells in situ. How to express new antigens in the disseminated tumor lesions, but not in normal tissue, have precluded the development of such strategies prior to this study.

NMD is an evolutionarily conserved surveillance mechanism in eukaryotic cells that prevents the expression of mRNAs containing a premature termination codon (PTC). Inhibition of NMD in cultured human cell lines using siRNA's targeted to any of its factors, SMG1, UPF1, UPF2 or UPF3, results in the upregulation of several products encoded by the PTC-containing mRNAs. Many of these products, resulting from aberrant splicing or NMD-dependent autoregulated alternative splicing encode new peptides that have not induced tolerance.

The hypothesis proposed herein, is that the upregulation of such products when NMD is inhibited in tumor cells will elicit an immune response against (at least some of) the new products, and that the immune response will inhibit tumor growth. Moreover, frameshift mutations in cancer cells exhibiting DNA mismatch repair generate PTC-containing transcripts may be negatively controlled by NMD. Inhibition of NMD could further augment the production of such tumor-specific antigens.

Materials and Methods

Tumor Immunotherapy Studies:

Three-hundred-thousand parental or pTIG-U6tetOshRNA transduced CT26 tumor cells were implanted subcutaneously in Balb/c or Nude mice. At the day of tumor implantation, mice started receiving water supplemented with 10% sucrose with or without 2 mg ml⁻¹ doxycycline (Sigma).

To evaluate the anti-tumor effects of PSMA aptamer-siRNAs, mice were implanted with 1×10⁶ PSMA-CT26 tumor cells and injected with 400 pmoles of aptamer-siRNA in 100 PBS via the tail vein at days 3, 5, 7, 9, 11 and 13. In combination therapy, treatment with PSMA aptamer-siRNA was administered at days 5, 7, 9, 11 and 13, and a single dose of 500 pmoles of 4-1BB aptamer dimer was administered on day 6.

To monitor metastasis, C57BL/6 mice were implanted with 10⁵ B16-PSMA transduced cells by the tail vein and injected with 400 pmoles of aptamer-siRNA conjugates at days 5, 8, 11, 14 and 17. When about half of the mice in the control groups had shown signs of morbidity (approximately days 25-28), the mice were euthanized and their lungs were weighed. GM-CSF-expressing B16/F10 tumor cells, were irradiated (50 Gy) and 5×10⁵ cells were injected subcutaneously at days 1, 4 and 7, or days 5, 8 and 11.

Statistical Analysis:

For statistical analysis, P values were calculated using a Student's t-test.

Results

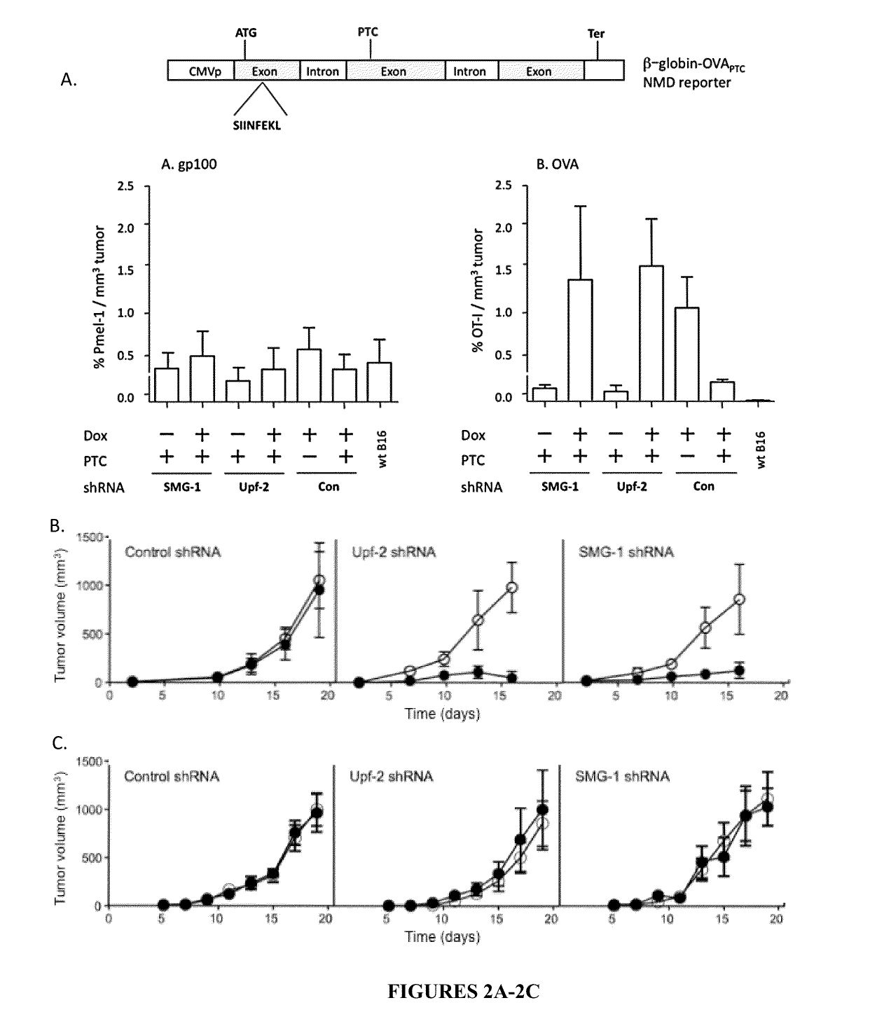

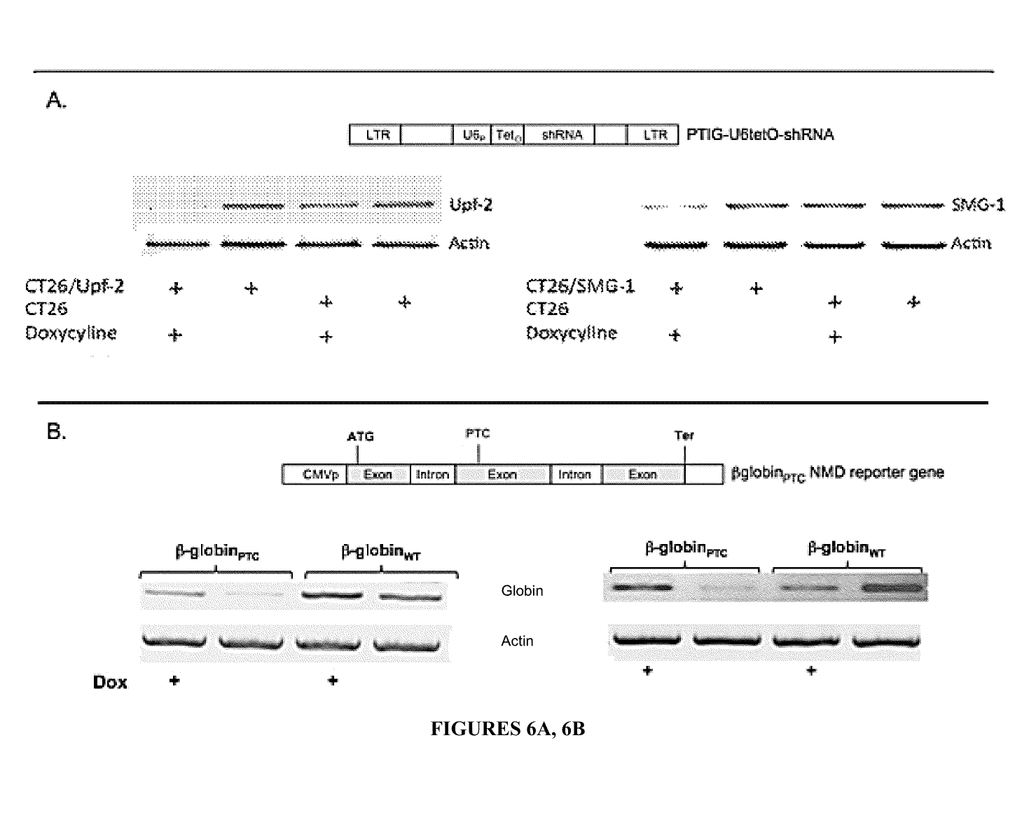

To determine whether NMD inhibition in tumor cells can stimulate protective anti-tumor immunity, it was tested whether the stable expression of NMD factor short hairpin RNAs (shRNAs) in tumor cells inhibits their growth potential in mice. CT26 colon carcinoma tumor cells were transduced with a lentiviral vector (PTIG-U6tetOshRNA) encoding Smg1 or Upf2 shRNAs expressed from a tet-regulated U6 promoter. shRNA expression can be upregulated in vitro by adding doxycycline to the culture medium, and in vivo by providing doxycycline in the drinking water. Doxycycline-induced Smg1 and Upf2 shRNA expression in cultured CT26 cells results in downregulation of the corresponding mRNA (FIG. 6A) and inhibition of NMD (FIG. 6B). Long-term inhibition of NMD, or other functions controlled by SMG1 or UPF2, had no measurable effects on the viability or proliferative capacity of the CT26 cells in vitro. To determine whether siRNA inhibition of NMD in the tumor-bearing mice can stimulate immune responses against products that are normally under NMD control, the intratumoral accumulation of T cells recognizing a model tumor antigen that is suppressed as a result of NMD, was measured. B16/F10 tumor cells containing the doxycycline-inducible Smg1, Upf2 or control shRNA were stably transfected with an NMD reporter plasmid encoding the dominant major histocompatibility complex (MHC) class I epitope of the chicken ovalbumin gene (OVA) upstream of a PTC (FIGS. 2A, 6A). Tumor-bearing mice were infused with OT-I transgenic CD8 T cells that recognize the OVA MHC class I-restricted epitope, or with Pmel-1 transgenic CD8 T cells that recognize an MHC class I-restricted epitope in the endogenous gp100 tumor antigen expressed in B16 tumor cells 19. gp100 expression is not under NMD control. As shown in FIG. 2A, unlike Pmel-1 T cells, the OT-I T cells failed to accumulate to significant levels in the OVA-negative B16/F10 tumors or in tumors transfected with the PTC-containing β-globin-OVA construct encoding but not expressing Smg1 or Upf2 shRNA. However, upregulation of Smg1 or Upf2 shRNA, but not control shRNA (doxycycline in the drinking water) resulted in a significant accumulation of OT-I T cells in the tumors. This experiment shows that siRNA inhibition of NMD in tumor cells can induce an immune response in vivo against an antigen that is under NMD control.

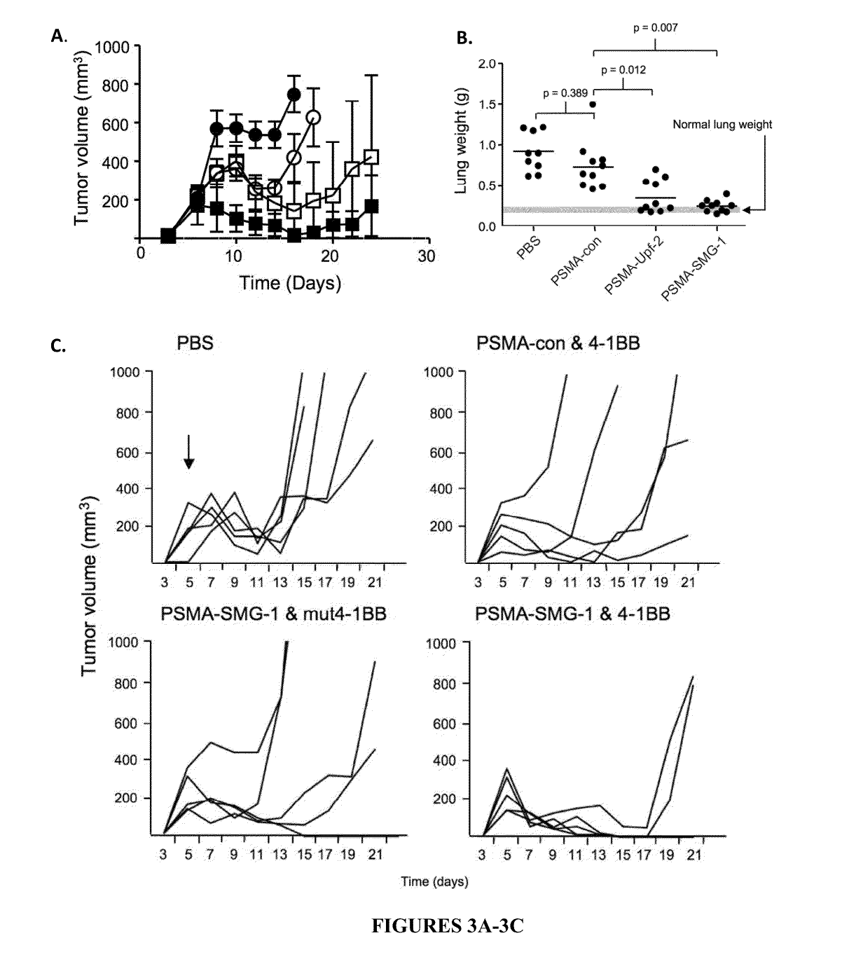

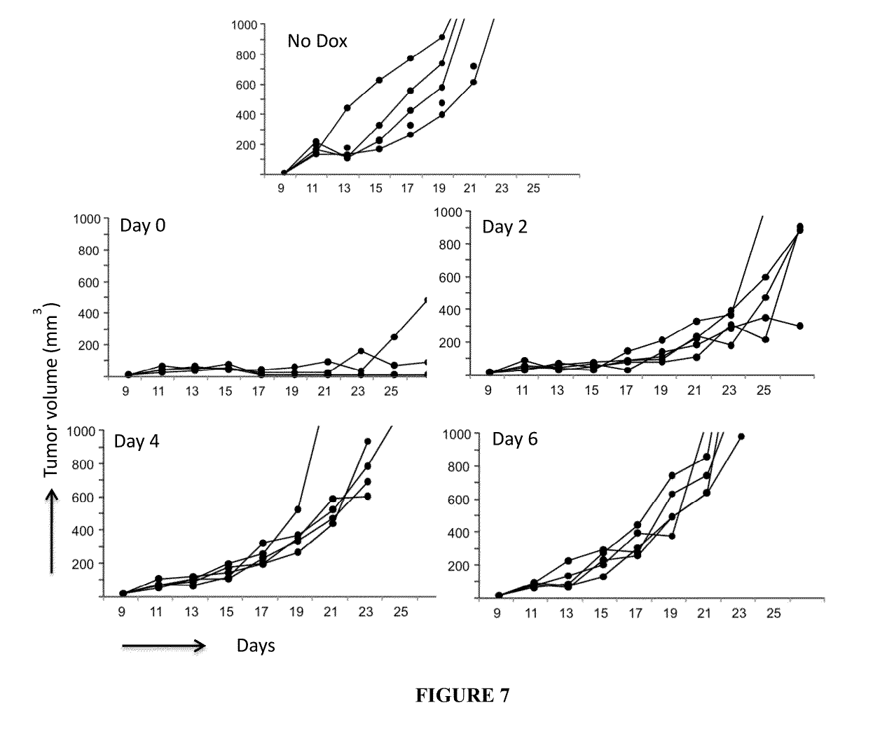

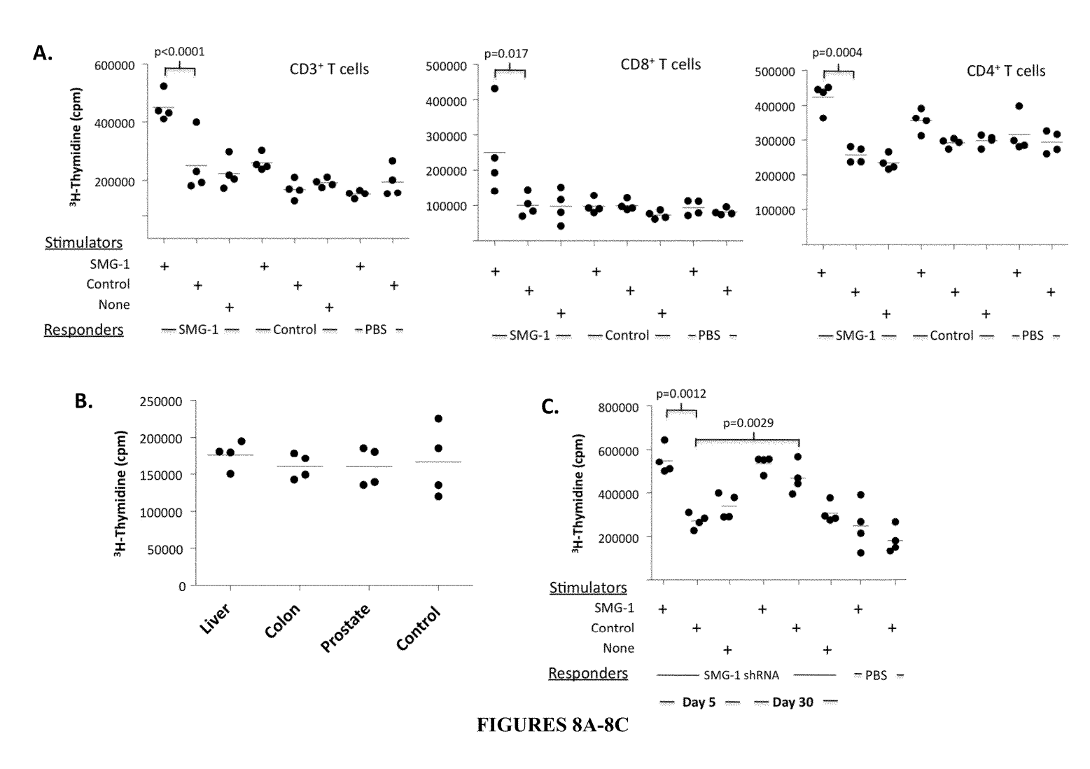

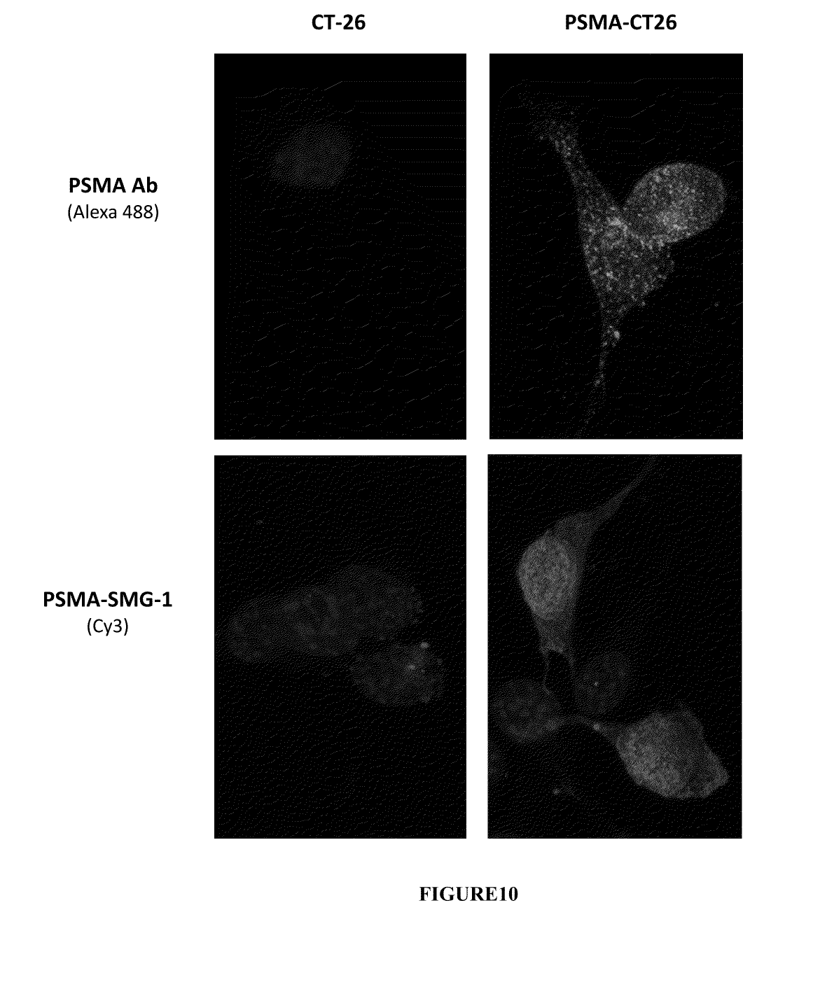

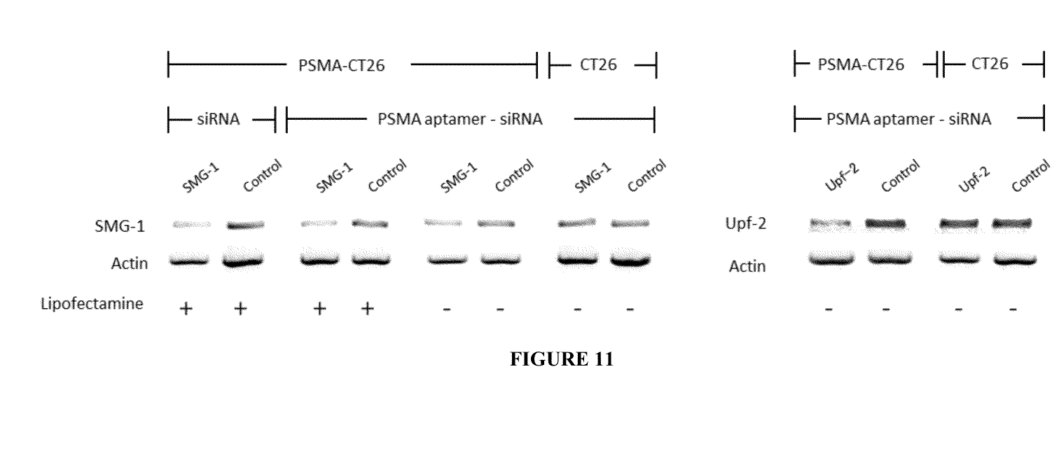

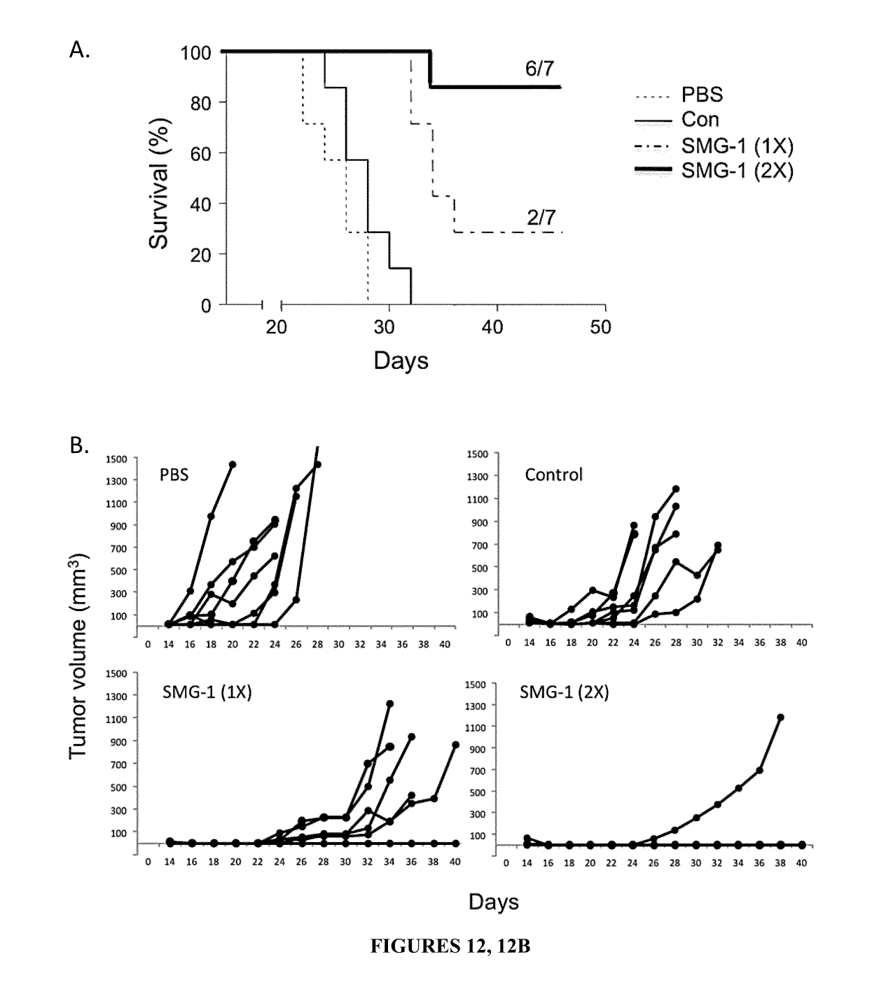

To determine whether siRNA-mediated inhibition of NMD affects tumor growth, the lentiviral-transduced CT26 cells expressing a control, Smg1 or Upf2 shRNA were implanted subcutaneously into mice and tumor growth was monitored in the presence or absence of doxycycline administered in the drinking water. FIG. 2B shows that tumor cells expressing Smg1 or Upf2 shRNA, but not control shRNA, grew initially but failed to progress. Tumor inhibition was immune-mediated because the tumors grew in nude mice (FIG. 2C), and mice that rejected the tumors shown in FIG. 2B, but not age-matched control mice, resisted a second challenge with parental tumor cells. Delaying doxycycline treatment of mice expressing Smg1 shRNA diminished the tumor inhibitory effect that was completely lost when drug treatment was delayed for 6 days (FIG. 7). Tumor rejection correlated with the induction of T-cell responses against tumor cells expressing Smg1 shRNA. No T-cell responses were detected against tumor cells that did not express Smg1 shRNA or against normal tissues including liver, colon and prostate (FIGS. 8A-8C). This is consistent with the hypothesis that tumor rejection was mediated by the induction of immune responses against NMD-controlled products that were upregulated when NMD was inhibited in the tumor cells. In the experiment shown in FIG. 2B, tumor growth was completely prevented when NMD was inhibited in all tumor cells from the time of tumor implantation. Simulating a more relevant clinical model, it was tested whether inhibition of NMD in pre-existing tumors can induce therapeutically useful tumor immunity. To preclude NMD inhibition in normal cells, the NMD factor siRNAs were targeted to tumor cells using oligonucleotide aptamer ligands. Smg1 and Upf2 siRNA were conjugated to an oligonucleotide aptamer that binds to prostate-specific membrane antigen (PSMA) as shown in FIG. 9. PSMA-expressing CT26 and B16 tumor cell lines were generated by transduction with a PSMA-encoding expression vector, and PSMA expression was confirmed by flow cytometry. The PSMA-conjugated siRNA's bound to and were taken up by PSMA-expressing, but not parental, tumor cells (FIG. 10), leading to the downregulation of their target RNAs (FIG. 11). It was next tested whether systemic administration of PSMA aptamer-siRNA conjugates by tail vein injection can inhibit tumor growth. As shown in FIG. 3A, treatment of day 3 subcutaneously implanted PSMA-CT26 tumor cells with PSMA-conjugated Smg1 siRNA, and to a lesser extent Upf2 siRNA, significantly inhibited tumor growth. Two out of seven mice treated with the PSMA aptamer-Smg1 siRNA conjugate rejected the implanted tumors and remained tumor-free (FIGS. 12A, 12B). When treatment intensity was increased by doubling the dose of the aptamer-siRNA conjugate and extending treatment to seven injections, six out of seven of mice rejected the tumor long term. Treatment with PSMA aptamer conjugated to control siRNA had a small inhibitory effect that could have resulted from the binding of the PSMA aptamer-siRNA to the tumor cells, or be due to non-specific immune stimulatory effects of the oligonucleotide. No increase was found in IFNα levels in the serum of mice treated with PSMA aptamer-control or Smg1 siRNA conjugates. As shown in FIG. 3B, the treatment of day 5 PSMA-B16/F10 tumor-implanted mice with PSMA aptamer-conjugated Upf2 or Smg1 siRNA inhibited the development of lung metastasis that was more profound in the SMG1 group. To determine whether the anti-tumor response elicited by NMD inhibition can be further enhanced by co-stimulation, PSMA-CT26 tumor-bearing mice were treated with PSMA aptamer-Smg1 siRNA and an agonistic 4-1BB aptamer dimer. The stringency of NMD inhibition and 4-1BB co-stimulation was adjusted to elicit a limited anti-tumor effect when applied separately by delaying treatment with PSMA aptamer-siRNA conjugates from days 3 to 5 and administering a single dose of 4-1BB aptamer on day 6. As shown in FIG. 3C, combination therapy with PSMA aptamer-Smg1 siRNA and 4-1BB aptamer was more than additive.

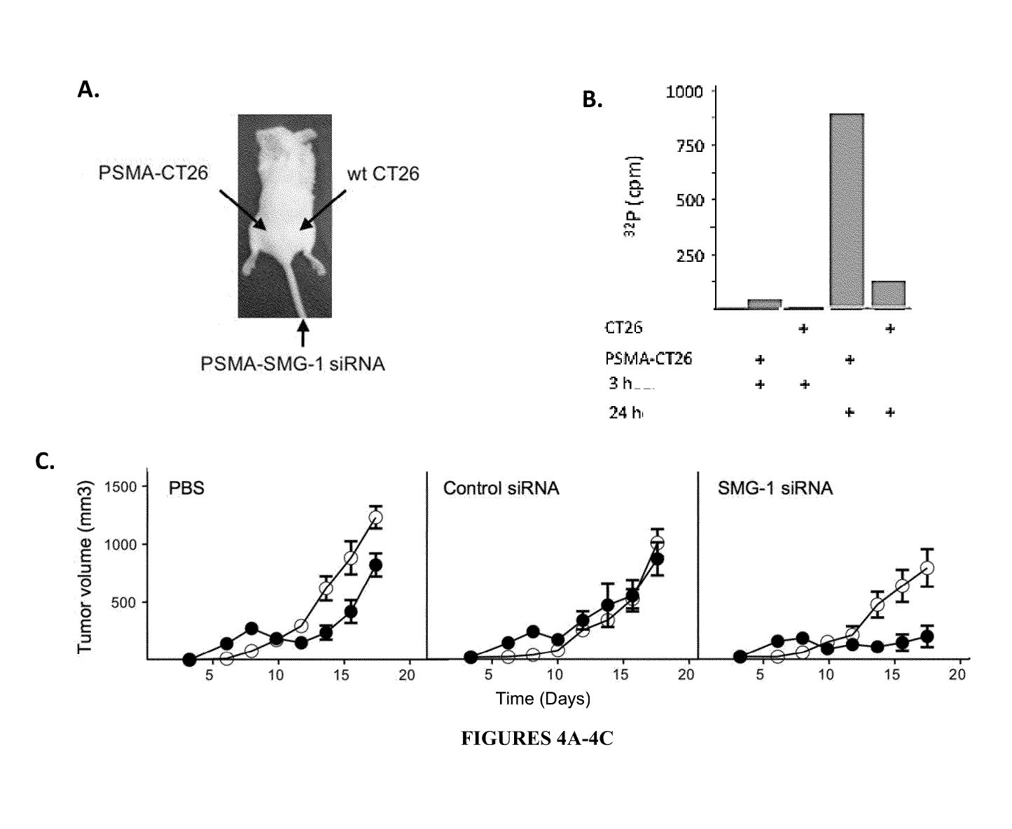

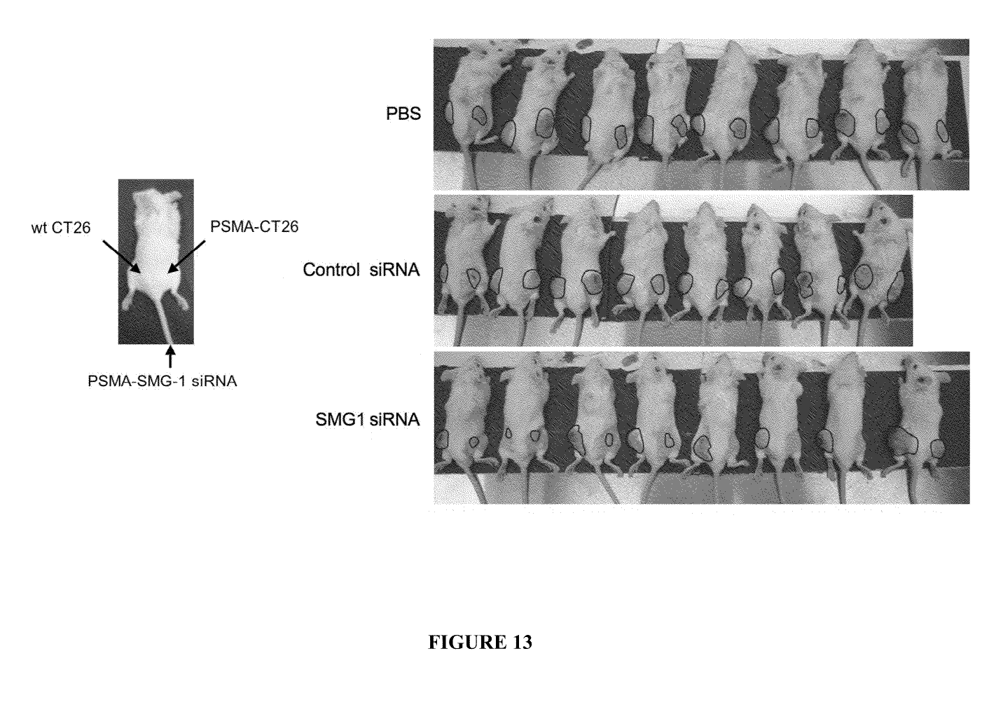

To determine whether tumor inhibition shown in FIGS. 3A-3C is a result of aptamer targeting of siRNA to PSMA-expressing tumor cells, mice were implanted in opposite flanks with PSMA-expressing and parental CT26 tumor cells and PSMA aptamer conjugated to control or Smg1 siRNA was administered systemically by tail vein injection (FIG. 4A). FIG. 4B shows that ³²P-labelled PSMA aptamer-Smg1 siRNA conjugate accumulated preferentially in PSMA-expressing tumor cells. FIG. 4C shows that systemic administration of PSMA aptamer-conjugated Smg1, but not control, siRNA inhibited the growth of PSMA-expressing CT26 tumor cells but not the contralaterally implanted parental CT26 tumor cells. FIG. 13 shows a snapshot of the tumor-bearing mice at the day of euthanization.

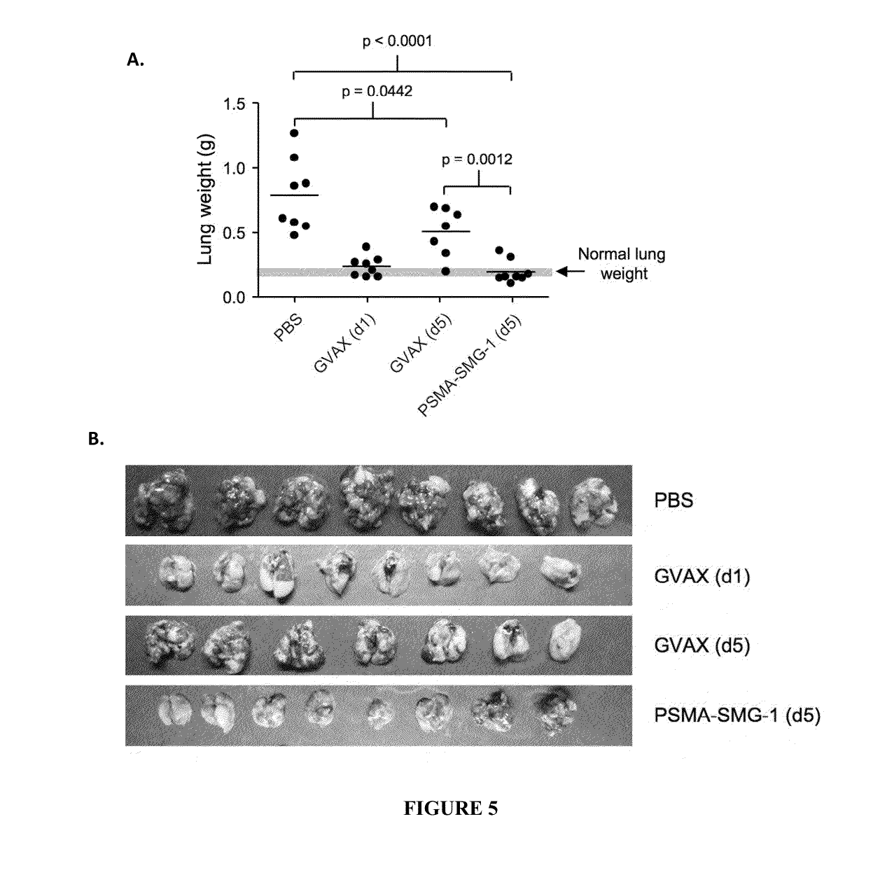

To assess the potency of tumor-targeted NMD inhibition, the anti-tumor effects of treating tumor-bearing mice with PSMA aptamer-Smg1 siRNA conjugate and vaccination with GM-CSF-expressing irradiated syngeneic tumor cells (GVAX) were compared. In therapeutic protocols when vaccination is initiated 2-4 days after tumor inoculation, the anti-tumor impact of GVAX is limited, unless combined with other treatments such as CTLA-4 blockade28 or T-regulatory cell depletion. As shown in FIG. 5, in the B16 lung metastasis model described in FIG. 3B, GVAX treatment of day 1 tumor bearing mice significantly inhibited metastasis, whereas treatment of day 5 tumor bearing mice had a limited anti-metastatic effect that barely reached statistical significance. By comparison, treatment of day-5 tumor-bearing mice with PSMA aptamer-Smg1 siRNAs inhibited metastasis to an extent comparable to that of administering GVAX at day 1. Given that these are first generation aptamer-siRNA conjugates and the dose and schedule of aptamer-siRNA treatment have not been optimized, these results indicate that tumor-targeted siRNA-mediated NMD inhibition is more effective than a commonly used vaccination protocol.

Induction of antitumor immunity against parental tumor-epitope spread: The mice induced to express SMG-1 or Upf-2 siRNA which rejected the tumors (FIG. 2B) were completely resistant to a subsequent challenge with parental tumor cells (7/7 mice). This is consistent with epitope spread whereby an initial immune response directed to antigens induced by Upf-2 or SMG-1 siRNA inhibition of NMD “spreads” to antigens expressed by the parental tumor. The underlying mechanism of “epitope spread” is that the immune response against the original antigen leads to the destruction of a proportion of the tumor targets, resulting in the release of endogenous antigens which are captured by local professional antigen presenting cells such as dendritic cells and presented to the immune system.

The observation that tumors in which NMD is inhibited elicit immune responses against the parental tumor appears, however, to be inconsistent with the results of FIG. 4C which shows that the PSMA aptamer targeted siRNA inhibition of tumor growth was local; it affected only the PSMA-expressing tumors but not the contralaterally implanted parental tumor cells. Clearly there was no evidence for epitope spread in this instance. A likely explanation that reconciles both observations is that the immune response induced by epitope spread to the endogenous (parental) tumor antigens is delayed and therefore was not detected when both PSMA-expressing and non PSMA-expressing tumor cells were implanted at the same time as was done in FIG. 4C.

To test this hypothesis, it was determined whether immune responses generated against the NMD controlled products as shown in FIG. 8A “spreads” to tumor antigens expressed in parental tumors. As shown in FIGS. 8C (and 4A), T cells isolated 5 days after implantation of SMG-1 shRNA expressing tumor cells, namely tumor cells in which NMD was inhibited, recognized NMD-inhibited, but not parental tumor cells, evidencing that the immune response was directed against the NMD controlled products which were upregulated upon NMD inhibition but not against endogenous antigens expressed in the parental tumor cells. It was hypothesized that if induction of immunity against the NMD products leads to epitope spread, at later time points the mice will generate immune responses also against the parental tumor. Indeed, as shown in FIG. 8C when T cell responses were measured 30 days post tumor inoculation an immune response was elicited against parental tumor cells which was comparable in magnitude to that elicited against the NMD controlled products. This experiment, therefore, provides immunological evidence that immune responses against NMD-controlled products “spreads” to endogenous tumor antigens and that it takes time to develop.

Tumor-targeted NMD inhibition is a new approach to stimulate protective anti-tumor immunity. Instead of stimulating or potentiating immune responses against existing, often weak, antigens expressed in the tumor cells—the goal of current tumor vaccination protocols—NMD inhibition generates new antigenic determinants in situ in the disseminated tumor lesions. It should be noted that NMD control of gene expression is ‘leaky’. In addition to the first round of translation, known as pioneer translation, the efficiency of nonsense-mediated degradation varies among individual mRNA targets. Immune recognition is, therefore, a consequence of upregulation of NMD-controlled products above a certain threshold that was set by the natural immune tolerance mechanisms. The NMD inhibition strategy described in this study comprises a reagent that can be synthesized by a cell-free chemical process, it obviates the need to identify TRAs or adjuvants, and is broadly applicable as it targets a common pathway in all tumors. The potency of the NMD inhibition approach was suggested when compared to GVAX vaccination. Arguably, these first generation aptamer-siRNA conjugates and the dose and treatment schedule can be further optimized. It would be of interest to determine in future studies whether the NMD-induced antigens are cross-reactive among different tumors, and if so to identify the dominant antigens induced by NMD inhibition.

### Example 2

### Synthesis and In Vitro Characterization of Peptide-siRNA Conjugates

Peptides & siRNA's:

siRNAs targeted to the murine NMD factors SMG-1 and Upf-2 are tested against three peptide ligands that bind to receptors upregulated in tumor endothelial cells and accumulate in the tumor vasculature:

(i). Neuropilin and Integrin Binding Peptide.

Using phage display library selection, a class of 9 amino acid long cyclic peptides, termed CendR peptides (prototype, c[CRGDRGPDC]; SEQ ID NO: 6), which combine binding motifs to both neuropilin and integrins. Both motifs contribute to the efficient binding of the peptide to tumor vasculature, while the neuropilin binding motif promotes the internalization of the bound ligand that is important for the intracellular delivery of the siRNA cargo.

(ii) Neuropilin & VEGR-1 Binding Peptide.

The short linear peptide, CPQPRLC (SEQ ID NO: 7), binds to VEGFR-1 and neuropilin-1 and targets endothelial cells in vivo.

(iii). BiP/Grp78 Binding Peptide.

Grp78 is a heat shock member of endoplasmic chaperones. In stressed cells, including endothelial cells in the tumor vasculature, a fraction of the intracellular grp78 is translocated to the cell surface. Short 7 to 11 amino acids long linear peptides (GIRLRG (SEQ ID NO: 4), WIFPWIQL (SEQ ID NO: 1) or YPHIDSLGHWRR (SEQ ID NO: 8)) which bind to grp78 on the cell surface will be used in these studies.

The integrinsneuropilin, and grp78, are highly conserved between mice and human. Indeed, the peptides chosen in this study were shown to bind to both murine and human orthologs on endothelial cells. Neuropilin and grp78, and to a lesser extent the integrins, are often upregulated also on tumor cells. This could further enhance siRNA delivery and its therapeutic impact when targeted to these receptors. The results will which of the receptors, integrins neuropilin and grp78, are upregulated on TRAMP-C1 or TRAMP-C2 tumor cells, both during in vitro culture and in vivo. If any of the receptors are expressed on the tumor cells, an experimental strategy to determine the extent siRNA targeting to the tumor vasculature or the tumor cells has contributed to the observed antitumor effect.

Peptide-siRNA Conjugation.

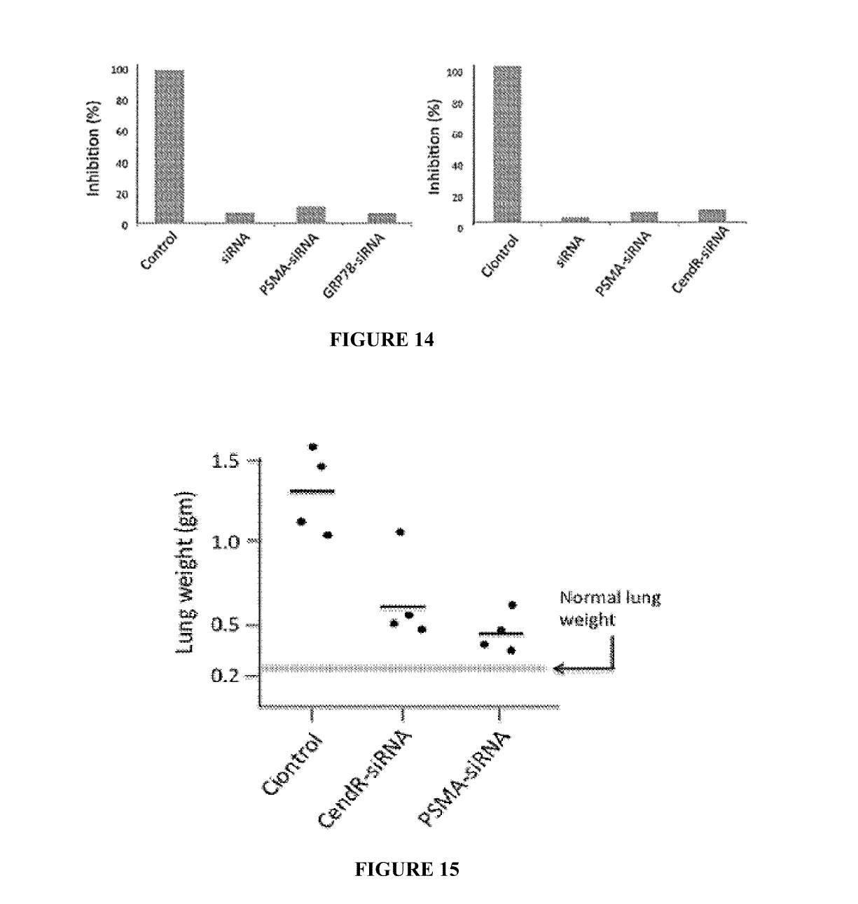

In preliminary studies, either of the two peptides, the neuropilin/integrin c[CRGDRGPDC] (SEQ ID NO: 6) peptide and the grp78/BiP GIRLRG (SEQ ID NO: 4) peptide, were conjugated to the passenger strand of SMG-1 siRNA. Standard chemistry was used to conjugate the primary amino group of the peptides thru a (glycol)₆ spacer to the thiol modified 5′ end of the siRNA passenger strand. In the second step the guide strand was hybridized to the peptide linked passenger strand. The composition of the conjugation products was confirmed by mass spectroscopy. To test whether conjugation has negatively impacted on siRNA function, the peptide-siRNA conjugates were transfected into HEK293 cells and target inhibition was measured. As shown in FIG. 14, in both instances peptide conjugation did not significantly impact on siRNA function (compare to free siRNA or PSMA aptamer conjugated siRNA).

Peptide-siRNA conjugations are carried out using alternative and advanced chemistries such as Click chemistry. In view of the notoriously low affinity of peptide ligands, in the low μmolar range compared to mid nanomolar range for aptamers, methods to generate bi- or oligovalent peptide conjugates using dendrimer wedges, termed Dendrons (Polymer Factory), which contain one monovalent functionalized group linked to an oligovalent (n=4, 8, 16, etc.) functionalized group will be explored. In addition, to evaluate the potential benefits of delivering multiple copies of siRNA “linear dendritic hybrids” are used which comprise two dendrimer wedges with distinct functionalized groups to accommodate multiple copies of both peptide and siRNA.

Binding of the Peptide-siRNA Conjugates to their Corresponding Target on the Cell Surface.

Since the peptide ligands bind to both murine and human orthologs expressed on EC, binding of the peptide-siRNA conjugates to HUVEC, an established human EC cell lines commonly used in peptide binding studies to endothelial cells is evaluated. Binding will be determined by flow cytometry using a 3′-flourophore (Alexa₄₈₈) tagged guide sequence hybridized to the peptide-passenger conjugate.

Since tumor cells often upregulate integrins, neuropilin and grp78 on the cell surface (the latter especially after irradiation) expression of the peptide targets, neuropilin, VEGR-1, integrins on the prostate tumor TRAMP-C1 and TRAMP-C2 cell lines are measured, as well as other tumor cell lines such as melanoma B16/F10, colon carcinoma CT26 or breast carcinoma 4T1, using flow cytometry and/or confocal microscopy. Specificity of binding is determined by either transfecting negative cells with an expression plasmid, or preferably by knocking down expression using shRNA in lentivirally transduced cells, as well as by using non-binding peptides.

Receptor Mediated (Peptide-Dependent) siRNA Inhibition of NMD.

siRNA inhibition of NMD is determined in two steps. In step I, conjugation of the siRNA to the peptide has not compromised its activity. This is done by comparing knockdown of an artificial target by free and conjugated siRNA using either the PSI-CHECK® dual luciferase system as shown in FIG. 14 or preferably using qRT-PCR to measure the knockdown of the endogenous SMG-1 and/or Upf-2 mRNA in transfectable murine cell lines (e.g., NIH 3T3 or Hepa) transfected with the peptide-siRNA (compared to free siRNA or PSMA aptamer-siRNA). In the event that the activity of the conjugated siRNA is compromised (not seen in 2/2 conjugates shown in FIG. 14), the peptide is conjugated to the 3′ end of the passenger sequence and/or a longer spacer is used.

Next, to determine NMD inhibition in a receptor-mediated, peptide-dependent fashion, the peptide-siRNA conjugates are incubated with cells in the absence of transfection reagent, using cells which express or do not express the cognate receptor as described above. Alternatively, blocking of binding is measured by competition with free peptide or antibody versus control peptides or antibodies.

Off Target Effects.

Since nucleic acid can elicit off target effects to stimulate the innate arm of the immune response, peptide-siRNAs which exhibit measurable levels of nonspecific immune stimulatory activity are excluded. This is determined by incubation with APC, e.g. adherence splenocytes or monocyte derived DC, and measuring secretion of inflammatory cytokines, IFNα, TNF and IL-6. The SMG-1 and Upf-2 siRNAs used in the studies have been previously shown to be devoid of non specific immune stimulatory properties both in vitro and in mice, and the corresponding peptide-siRNAs used in FIG. 14 also failed to exhibit off-target effects in vitro.

### Example 3

### Evaluation of the Antitumor Activity of the Peptide-siRNA Conjugates in the TRAMP-C Model for Prostate Cancer

The TRAMP-C Model.

The transgenic adenocarcinoma mouse prostate (TRAMP) mice harbor a construct comprised of the minimal rat probasin promoter driving prostate specific epithelial expression of the SV40 large T antigen. The TRAMP model mirrors the pathogenesis of human prostate cancer whereby male TRAMP mice uniformly and spontaneously develop orthotopic prostate tumors consequent to the expression of SV40 T antigen. The TRAMP-C lines were derived from a heterogeneous 32 week tumor of a TRAMP mouse. The TRAMP-C1 and TRAMP-C2, but not TRAMP-C3, lines are tumorigenic when transplanted into syngeneic C57BL/6 mice. Both TRAMP-C1 and TRAMP-C2 lines obtained from the ATCC are used in these studies. The use of the TRAMP-C1 and/or the TRAMP-C2 lines is dictated by the expression of the peptide targets as discussed above. C57BL/6 mice are implanted subcutaneously with TRAMP-C cells and treated by tail vein injection with peptide-siRNA conjugate starting 3, 7 or 12 days post tumor implantation. The ability to delay treatment while observing a therapeutic impact is taken as an indication of the potency of the peptide-siRNA mediated antitumor activity. The treatment regimen, dose and frequency, is determined experimentally, guided by previous experience with PSMA aptamer-siRNA conjugates that were implanted 3 to 5 days post implantation of CT26 or B16/F10 tumors and treated with 500 pmoles of aptamer-siRNA conjugate 5 times every second day. The objective of these studies is to identify a best-in-class peptide-siRNA conjugate in terms of inhibiting TRAMP-C tumor growth in the absence of significant toxicity.

In preliminary studies the antitumor activity of the CendR c[CRGDRGPDC] (SEQ ID NO: 6) peptide-SMG-1 siRNA conjugate was evaluated in (PSMA-expressing) B16/F10 melanoma tumor model. In the experiment shown in FIG. 15, 5 day old tumor bearing mice implanted with PSMA-expressing B16/F10 melanoma cells were treated with either PSMA aptamer-SMG-1 siRNA conjugate, the positive control as described (Pastor, F., et al., Nature 465:227-231 (2010)), or with CendR peptide-SMG-1 siRNA conjugate. Both PSMA aptamer and peptide-targeted conjugates inhibited metastasis. The reduced antitumor effect of the peptide-siRNA conjugate compared to PSMA aptamer-siRNA conjugate (which was not statistically significant) could reflect suboptimal configuration or more likely its small molecular weight which is below the kidney filtration cutoff.

An experimental design similar to the one shown in FIG. 15 is used to evaluate the antitumor efficacy of tumor targeted NMD inhibition with peptide-siRNA conjugates; the major difference being that the TRAMP-C tumors are implanted subcutaneously since they do not appear to metastasize. Should any results show otherwise the experimental system is modified accordingly. In these experiments the relative potency of the peptide-siRNA conjugate compared to PSMA aptamer-siRNA conjugate treatment is compared and either of two or GM-CSF-expressing irradiated TRAMP-C tumor cells that are generated by transfection of a GM-CSF expression plasmid into TRAMP-C tumor cells. Of note, the PSMA aptamer-SMG-1 siRNA conjugates were superior to GM-CSF-expressing irradiated tumor cell vaccines in the B16 metastasis model.

To determine the contribution of siRNA targeting to endothelial cells versus targeting to tumor (when the receptor for the peptide ligand is expressed on both tumor and tumor EC), the antitumor activity of peptide and PSMA aptamer conjugated siRNAs are compared in mice implanted with the TRAMP-C tumor cells expressing or not expressing the cognate receptor. Lastly, the generality of using peptide targeted-siRNA delivery and tumor-restricted NMD inhibition are assessed in other tumor models, melanoma B16/F10, colon carcinoma CT26 and breast carcinoma 4T1 tumor cells. As a control, PSMA aptamer-siRNA conjugate is used. The hypothesis is that the peptide-siRNA conjugate will exhibit antitumor activity in all tumor models whereas the PSMA aptamer-siRNA conjugate will be effective only in tumor cells engineered to express PSMA.

Toxicity Studies.

General toxicity is evaluated by monitoring mice for treatment-associated mortality and morbidity. Angiogenesis-related adverse effects—the EC receptors targeted in this study are also upregulated during normal angiogenesis—are evaluated by measuring delay in wound healing or reduced litter size as described previously (Nair, S., et al., Blood 102:964-971(2003)).

Bioavailability.

The small MW of the peptide-siRNA conjugates (7-10 Kdal) is below the cutoff of kidney filtration (25-50 Kdal). Conjugation of siRNAs (Rusconi, C. P., et al., Nat Biotechnol 22:1423-1428 (2004)) and aptamers (Dassie, J. P., et al. Nat Biotechnol 27:839-849 (2009)) to carriers can significantly enhance their plasma half-life which was accompanied by a significant improvement of biological activity. It is therefore highly likely that the biological activity of the peptide-siRNA conjugates as indicated in FIG. 15 can be significantly enhanced by increasing their size and thereby promoting their retention in the circulation. To enhance the circulation half life and bioavailability of the peptide-siRNA conjugates cholesterol or polyethylene glycol carriers are to be used. Carriers are covalently attached through glycol spacers to the 5 end (or alternatively to the 3′ end) of the passenger strand. The PK/PD of the free and carrier modified peptide-siRNA conjugates are determined using ³²P-labeled peptide-siRNA (PK) as we have previously described (Pastor et al., 2010) and dose dependent tumor inhibition (PD).

Mechanistic Studies.

Without wishing to be bound by theory, the hypothesis is that the peptide-siRNA mediated inhibition of tumor growth is a result of (i) tumor vasculature (and tumor when applicable) targeting, (ii) siRNA action, and (iii) an adaptive antitumor immune response.

Tumor & Tumor Vasculature Targeting.

³²P-labeled and fluorophore-labeled peptide-siRNA (in the guide strand) are used to measure accumulation of the systemically injected peptide-siRNA in the tumor and the tumor vasculature, by counting radioactivity in the tumor and by confocal microscopy, respectively. Differential accumulation in the TRAMP-C tumor cells versus endothelial or other stromal components is determined by multiparameter flow cytometry for CD33 (EC marker), SV40 T antigen (tumor cell marker) and the flurophore used to label the peptide-siRNA conjugate (e.g. Alexa₄₈₈ or any other newer alternatives).

siRNA Mediated Action and Off-Target Effects.

To demonstrate that tumor inhibition is mediated through the action of the conjugated siRNA: (i) use of peptides-conjugated to control scrambled siRNAs, (ii) use of RACE-PCR on tumor RNA to identify the predicted cleavage products, and (iii) evaluate the potential contribution of non specific immune effects by measuring the levels of TNF, IFNα, and IL-6 in the circulation.

The Role of Immunity.

The key role of an adaptive immune response in mediating tumor rejection following targeted inhibition of NMD in the tumor vasculature is demonstrated: (i). Determining if tumor rejection is compromised in immune deficient nude mice. (ii). Antibody depletion of CD8 and CD4 T cell subsets. (iii). Measuring CD4 and CD8 proliferative responses against endothelial targets VEGFR2 and Tie2 and tumor-expressed antigens SV40 T antigen and TERT. Immunity to tumor-expressed antigens is postulated to be generated if the neuropilin and/or grp78 receptors are expressed on the tumor cells, but also, albeit with a delay, as a result of epitope spread.

### Example 4

### To Determine Whether Treg Depletion, CTLA-4 Blockade, and 4-1BB Costimulation, Will Synergize with NMD Inhibition to Potentiate Antitumor Immunity

The propensity of tumors to elaborate a host of mechanisms to frustrate their immune mediated elimination is thought to represent a 7th hallmark of cancer. Without wishing to be bound by theory, it is hypothesized that the antitumor immune response induced against the NMD-controlled products may not suffice to reverse tumor progression, especially in advanced cancer patients with significant tumor burdens. This might also most apply to the highly relevant, spontaneous TRAMP model for prostate cancer in which instance, multiple attempts of immunological interventions, initiated when tumor growth became evident, were able to slow tumor progression and prolong survival but were not curative (Cipriani, B., et al. 2008. Hum Gene Ther 19:670-680; Nanni, P., et al. 2007. Cancer Res 67:11037-11044; Nava-Parada, P., et al., 2007. Cancer Res 67:1326-1334; Quaglino, E., et al. 2004. The Journal of Clinical Investigation 113:709-717). Three promising immune stimulatory strategies are used to determine if they will synergize with the NMD inhibition approach to enhance the immune response generated against the newly expressed antigens and provide superior control of tumor growth. These studies will guide the development of immune-based interventions in the spontaneous TRAMP model and cancer patients. The three immune stimulatory strategies explored in these studies are elimination of Treg, CTLA-4 blockade, and 4-1BB costimulation:

Elimination of Foxp3-Expressing Regulatory T Cells.

Foxp3-expressing CD25^{|} CD4^{|} regulatory T cells (Treg) play a key role in attenuating tumor immunity and limit the efficacy of antitumor vaccination strategies. Elimination of Treg in mice by treatment with a depleting anti-CD25 antibody synergizes with vaccination to enhance the immune-mediated rejection of tumor, including poorly immunogenic tumors when used in combination with a vaccination protocol. Animal studies indicate that transient and partial reduction in Treg numbers in cancer patients could be used to enhance the potency of vaccine-induced antitumor immunity. The potential benefits of depleting Treg in cancer patients was also indicated in phase I/II clinical trials. Elimination of Treg in renal cancer patients with an IL-2-diphteria toxin fusion (ONTAK®) resulted in a 3-5 fold enhancement of tumor-specific T cell responses (Dannull, J., et al. 2005. J Clin Invest 115:3623-3633) and in a second study ONTAK® treatment of CEA positive breast and colorectal patients was accompanied by elevated CEA-specific T cell responses (Morse, M. A., et al. 2008. Blood 112:610-618). Transient and partial reduction in Treg numbers in cancer patients could, therefore enhance the potency of vaccine-induced antitumor immunity.

CTLA-4 Blockade.

CTLA-4 is a coinhibitory molecule which negatively regulates T cell activation. CTLA-4 is upregulated on activated T cells and is constitutively expressed on foxp3-expressing Treg. The therapeutic potential of manipulating CTLA4 function was demonstrated in murine models showing that systemic administration of a blocking anti-CTLA4 antibody (CTLA4 blockade) led to the rejection of transplanted tumors, including poorly immunogenic tumors if combined with vaccination or chemotherapy. Treatment with the anti-CTLA-4 antibody was associated with mild autoimmune manifestations conceivably representing the activation of autoreactive T cells.

4-1BB Costimulation.

4-1BB is a major costimulatory receptor upregulated on antigen-activated T cells whereas its ligand, 4-1BBL is upregulated on activated DC and B cells. 4-1BB signaling promotes primarily the survival and proliferation of antigen-activated CD8⁺ T cells in vitro and in vivo. Since CD8 T cells play a pivotal role in tumor immunity, enhancing 4-1BB costimulation could promote the generation of protective antitumor immune responses. In mice, tumor cells engineered to express 4-1BBL, a single chain anti-4-1BB antibody, or systemic administration of agonistic anti-4-1BB antibodies, enhanced tumor immunity and tumor rejection. An oligonucleotide aptamer which bound to murine 4-1BB expressed on the cell surface of activated CD8 T cells, and in its dimeric or multimeric form was capable of activating CD8 T cells and enhance vaccine-induced protective immunity in murine tumor models. Notably, on a molar basis the 4-1BB aptamer was as effective as treatment with an agonistic 4-1BB antibody (McNamara, J. O., et al., 2008. J Clin Invest 118:376-386.).

Co-treatment of tumor bearing TRAMP-C1 mice with peptide-siRNA conjugate and any of the three immune stimulatory strategies described above. Inhibition of tumor growth is compared to monotherapy with conjugate or immune stimulatory agent alone. For CTLA-4 blockade and Treg depletion monoclonal antibodies to CTLA-4 and CD25 are used respectively, whereas for 4-1B costimulation dimeric 4-1BB aptamers are used.

Future Experiments:

(1) Evaluate the antitumor potential in the highly relevant spontaneous TRAMP model for prostate cancer. The goal is to demonstrate that tumor vasculature targeted NMD inhibition in combination with any of the immune-stimulatory protocols to be tested, exhibits a superior antitumor effect in the TRAMP model compared to published protocols. (2) Develop and in vitro characterize human reagents, EC targeting peptide conjugated to a best-in-class human NMD factor specific siRNA. (3) Develop a protocol and generate GMP-grade material. (4) Carry out a phase I clinical trial in patients with hormone refractory prostate cancer to establish the safety of administering tumor vasculature homing peptide-siRNA conjugates, and secondarily to obtain indication of immunological and/or clinical responses. (5) Carry out a phase I/II combination clinical trial with peptide-siRNA conjugate and an immune stimulatory reagent.

The Abstract of the Disclosure is provided to allow the reader to quickly ascertain the nature of the technical disclosure. It is submitted with the understanding that it will not be used to interpret or limit the scope or meaning of the following claims.

What is claimed:

1. A composition for inhibiting nonsense mediated decay (NMD) pathways in patients in vivo comprising at least one first domain which specifically binds to at least one tumor cell target or normal cell target in a tumor stroma and at least one second domain specific for a molecular component of nonsense mediated decay pathways, wherein the second domain comprises an antisense oligonucleotide molecule, peptides, proteins, nucleic acids, organic or inorganic molecules, which inhibit the nonsense mediated decay pathway.

2. The composition of claim 1, wherein the oligonucleotide molecule of the second domain, comprising at least one of a short interfering RNA (siRNA); a micro-interfering RNA (miRNA); antisense oligonucleotides; a small, temporal RNA (stRNA); a short, hairpin RNA (shRNA), or combinations thereof.

3. The composition of claim 1, wherein the oligonucleotide molecule of the second domain, inhibits function and/or expression of at least one factor associated with the NMD pathway comprising at least one of: RENT1, RENT2, eIF4A, UPF1, UPF2, UPF3B, RNPS1, Y14, MAGOH, NMD1, SMG, or combinations thereof.

4. The composition of claim 1, wherein the first domain specifically binds to tumor or normal cell targets in tumor stroma, comprising: vascular endothelial growth factor (VEGF), vascular endothelial growth factor receptor (VEGFR-2), Tie2; fibronectin, vitronectin, collagen, laminin, fibroblast antigens, fibroblast activation protein (FAP), glucose-regulated protein 78 (GRP78), stromal derived factor 1 (SDF-1), stromal derived factor 2 (SDF-2), MCP-1, MIP-1α, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, neurophilin, MAdCAM-1, neuropilin-1, α4 integrins, α5 integrins, or beta defensins 3 and 4.

5. The composition of claim 4, wherein the molecules comprising the first domain comprises: an aptamer, a multimer (aptamer), an antibody or fragments thereof, synthetic molecules, small molecules, peptides, peptides having a C-terminal arginine (CendR) motif or C-terminal lysine motif, glucose-regulated protein 78 (GRP78) binding peptides, integrin binding peptides, Pie42 peptides, or combinations thereof.

6. The composition of claim 4, wherein the molecules comprising the first domain comprise at least one peptide set forth as SEQ ID NOS: 1, 2, 3, 4, 5, 6, 7, 8, or combinations thereof.

7. The composition of claim 1, wherein the first and second domains further comprise one or more domains to promote intracellular delivery, cytoplasmic delivery, bioavailability, or combinations thereof.

8. The composition of claim 5, wherein the one or more domains, comprise at least one of: polylysine, polyarginine, Antennapedia-derived peptides, HIV derived tat peptide, a fusogenic peptide from influenza hemagglutinin protein, a 9mer Arg oligopeptide, peptide transporters, intracellular localization domain sequences, or combinations thereof.

9. The composition of claim 5, wherein the one or more domains promoting bioavailability comprising at least one of: cholesterol, polyethylene glycol, or combinations thereof.

10. The composition of claim 1, wherein the first domain is an aptamer specific for the target molecules and the second domain is an siRNA specific for an NMD target.

11. The composition of claim 1, wherein the at least one first domain are fused or linked to each other and/or with the second domain by at least one linker molecule.

12. The composition of claim 11, wherein said linker molecule comprising: nucleotide, non-nucleotide, or mixed nucleotide/non-nucleotide molecules.

13. The composition of claim 11, wherein the one or more linker molecules comprising about 2 nucleotides length up to about 50 nucleotides in length.

14. The composition of claim 11, wherein the non-nucleotide linker comprises abasic nucleotide, polyether, polyamine, polyamide, peptide, carbohydrate, lipid, polyhydrocarbon, polymeric compounds or combinations thereof, and having one or more monomeric units.

15. A composition for inducing novel antigens in abnormal cells, comprising: a multi-domain molecule having at least one target specific domain and at least one domain, which modulates expression and function of molecules associated with nonsense mediated decay pathways.

16. A method of inducing or enhancing immunogenicity of a target cell in vitro or in vivo and modulating an antigen specific immune response in patient comprising:
obtaining a composition comprising at least target binding molecule conjugated to at least one oligonucleotide molecule wherein the target binding ligand selectively binds to tumor cell or normal cell target ligands of tumor stroma and the oligonucleotide is specific for a molecule associated with at least one factor associated with a nonsense mediated decay pathway (NMD);
administering the composition in a therapeutically effective amount to the patient; and,
inducing or enhancing the immunogenicity of a target cell and modulating an antigen specific immune response in patient.

17. The method of claim 16, wherein the oligonucleotide molecule of the second domain comprising at least one of a short interfering RNA (siRNA); a micro-interfering RNA (miRNA); antisense oligonucleotide; a small, temporal RNA (stRNA); a short, hairpin RNA (shRNA), or combinations thereof.

18. The method of claim 16, wherein the oligonucleotide molecule of the second domain inhibits at least one factor associated with the NMD pathway comprising at least one of: RENT1, RENT2, UPF1, UPF2, UPF3B, RNPS1, Y14, MAGOH, NMD1 or SMG.

19. The method of claim 16, wherein target binding ligand selectively binds at least one of: vascular endothelial growth factor (VEGF), vascular endothelial growth factor receptor (VEGFR-2), Tie2; fibronectin, vitronectin, collagen, laminin, fibroblast antigens, fibroblast activation protein (FAP), glucose-regulated protein 78 (GRP78), stromal derived factor 1 (SDF-1), MCP-1, MIP-1 MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, neuropilinMAdCAM-1, neuropilin-1, α4 integrins, α5 integrins, or beta defensins 3 and 4.

20. The method of claim 16, wherein the target binding ligand comprises at least one peptide having a C-terminal arginine (CendR) motif or C-terminal lysine motif, glucose-regulated protein 78 (GRP78) binding peptides, integrin binding peptides, or Pie42 peptides.

21. The method of claim 16, wherein the target binding ligand comprises at least one motif: RGD, R/KXXR/K, CX_{N+1}CX_{N+1}CX_{N+1}C motif, wherein X is a variable amino acid and n=0 to 50, variants, mutants or analogs thereof.

22. The method of claim 16, wherein the target binding ligands comprising the first domain comprise at least one peptide set forth as SEQ ID NOS: 1, 2, 3, 4, 5 or combinations thereof.

23. The method of claim 16, wherein target binding ligands comprise peptides which bind to tumor vasculature or tumor stroma are identified by phage display libraries or high-throughput screens.

24. The method of claim 16, wherein the target binding ligand is an aptamer.

25. The method of claim 16, further comprising administering an immune cell co-stimulatory agent comprising wherein the agent is specific for immune cell stimulatory molecules comprising at least one of: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

26. A method of up-regulating existing and/or inducing new or novel antigens on a cell's surface comprising contacting the cell with the composition comprising at least one first domain which binds to at least one tumor or normal cell target ligand in tumor stroma and at least one second domain specific for a molecular component of nonsense mediated decay pathways, wherein the NMD domain comprises an antisense oligonucleotide molecule, peptides, proteins, nucleic acids, organic or inorganic molecules which inhibit the nonsense mediated decay pathway.

27. A method of preventing or treating cancer in vivo, comprising administering to a patient a therapeutically effective amount of a composition comprising at least one first domain which binds to at least one tumor cell or normal cell target ligand in tumor stroma and at least one second domain specific for a molecular component of nonsense mediated decay pathways, wherein the NMD domain comprises an antisense oligonucleotide molecule, peptides, proteins, nucleic acids, organic or inorganic molecules which inhibit the nonsense mediated decay pathway.


SEQUENCE LISTING (8 sequences)

SEQ ID NO: 1 - protein, 8 aa, Homo sapiens
WIFPWIQL

SEQ ID NO: 2 - protein, 12 aa, Homo sapiens
WDLAWMFRLPVG

SEQ ID NO: 3 - protein, 13 aa, Homo sapiens
CTVALPGGYVRVG

SEQ ID NO: 4 - protein, 6 aa, Homo sapiens
GIALRG

SEQ ID NO: 5 - protein, 6 aa, Artificial Sequence
  VARIANT 3, 4: Xaa = Any Amino Acid
RKXXRK

SEQ ID NO: 6 - protein, 9 aa, Artificial Sequence
  synthetic peptide
CRGDRGPDC

SEQ ID NO: 7 - protein, 7 aa, Artificial Sequence
  synthetic peptide
CPQPRLC

SEQ ID NO: 8 - protein, 12 aa, Artificial Sequence
  synthetic peptide
YPHIDSLGHWRR